SEQUENCE LISTING (1 sequence)

SEQ ID NO: 1 - DNA, 608 bp, Artificial Sequence
  synthetic
tgcctgggtaaagaagctagggaattgggggatatggacatagagtgatcatgtttattctctttccaacaaaggcctgaattaacaaacacaaaatggctctgctgctaagaaaaacacagtgggtagatacacttgaaagtcacttctgaaagagaccttgaagaagggatggagaatgtttggggtggacagaaactcctccaagaaagacaatgaaagacaacaagttaggtcacggtggctaaagaggagacaatgatttgttgtctgactctccagctacgatggggctgtaactaatgaggttattctggttacaaaggaagcattcagccccttaatgaaaaactctttggcaactggtgttagggaaagaaaaaacaaaccagcagcttattattagttccacagcaagttaattaatttttgccagtcactttgggtcacttgctggactgagcatctgtctcactcaactgaacagctggagggatggtgggtgtctctgcgggccatgcatgtgtcctgttccgggaggagaagggagagctaactacaaggctgaaaaaacgtgagcttccagtgttcatctgctcctctcctgt

(19) United States
(12) Patent Application Publication
(10) Pub. No.: US 2023/0015647 A1
(43) Pub. Date: Jan. 19, 2023

(54) INHIBITORS OF HISTONE DEMETHYLASES (PFI-63 AND PFI-90) FOR THE TREATMENT OF CANCER AND FOR THE INHIBITION OF HISTONE DEMETHYLASE IN CELLS

(71) Applicant: The United States of America,as represented by the Secretary,Department of Health and Human Services, Bethesda, MD (US)

(72) Inventors: Javed Khan, Rockville, MD (US); Robert G. Hawley, Bethesda, MD (US); Girma M. Woldemichael, Bethesda, MD (US); Megan L. Peach, Frederick, MD (US)

(73) Assignee: The United States of America,as represented by the Secretary,Department of Health and Human Services, Bethesda, MD (US)

(21) Appl. No.: 17/777,552

(22) PCT Filed: Nov. 18, 2020
(86) PCT No.: PCT/US2020/060955

§ 371 Date: May 17, 2022

Related U.S. Application Data
(60) Provisional application No. 62/936,722, filed on Nov. 18, 2019.

(51) Int. Cl. A61K 31/4402

(52) U.S. Cl. CPC A61K 31/4402

(57) ABSTRACT

The invention provides methods and compositions involving the compounds N′-(3,5-dimethylbenzoyl)picolinohydrazide; N′-(pyridin-2-yl)picolinohydrazide or pharmaceutically acceptable salts thereof for inhibiting the function of histone demethylases in vivo and in vitro, as well as methods for treating cancer comprising the administration of such compositions.

### CROSS REFERENCE TO RELATED APPLICATIONS

This application claims the benefit of U.S. Provisional Patent Application No. 62/936,722, filed Nov. 18, 2019, the disclosure of which is incorporated by reference in its entirety.

### STATEMENT REGARDING FEDERALLY SPONSORED RESEARCH OR DEVELOPMENT

This invention was made with Government support under Grant Number ZIA BC 010806 awarded by the NCI Intramural Program. The government holds certain rights in this invention.

### INCORPORATION-BY-REFERENCE OF MATERIAL SUBMITTED ELECTRONICALLY

Incorporated by reference in its entirety herein is a computer-readable nucleotide sequence listing submitted concurrently herewith and identified as follows: one 1,281 byte ASCII (Text) file named “750948_ST25.TXT,” created on Nov. 17, 2020.

### BACKGROUND OF THE INVENTION

Histone demethylases are enzymes that remove methyl groups from histone proteins. The enzymes alter transcriptional regulation by controlling methylation levels in histones and, in turn, regulate the chromatin state at specific gene loci. These enzymes play a role in epigenetic modification mechanisms. Specifically, histone demethylases play a role in the epigenetic landscape of the chromatin environment in various cancer cells.

Histone demethylases are therefore a possible target for the treatment of cancer. Accordingly, compositions and methods that may be used to modify the function of histone demethylases are needed.

### BRIEF SUMMARY OF THE INVENTION

The invention provides a method of treating cancer in a mammal. The method comprises, consists essentially of or consists of administering an effective amount of one or more compounds selected from the group consisting of:

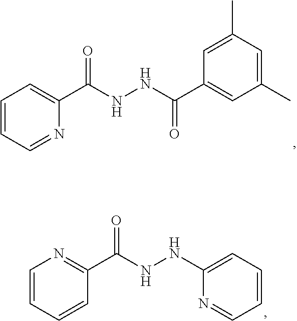 [SMILES: Cc1cc(C)cc(C(=O)NNC(=O)c2ccccn2)c1 ; O=C(NNc1ccccn1)c1ccccn1]

and pharmaceutically acceptable salts thereof, to the mammal.

The invention also provides a method for inhibiting a histone demethylase in cells comprising, consisting essentially of or consisting of contacting the cells with one or more compounds selected from the group consisting of:

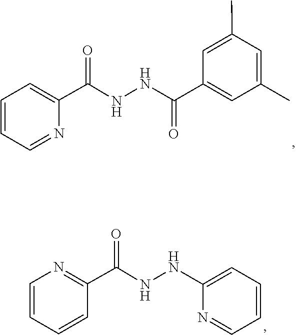 [SMILES: Cc1cc(C)cc(C(=O)NNC(=O)c2ccccn2)c1 ; O=C(NNc1ccccn1)c1ccccn1]

and pharmaceutically acceptable salts thereof.

### BRIEF DESCRIPTION OF THE SEVERAL VIEWS OF THE DRAWING(S)

FIG. 1 is a visual representation of the pGreenFire green fluorescent protein (GFP)-luciferase reporter sequence (SEQ ID NO: 1) used in a high throughput screen for identifying compounds that disrupt the function of the PAX3-FOXO1 fusion transcription factor in RH4 fusion positive rhabdomyosarcoma cells (see Gryder et al., Cancer Discov. 7: 884-899 (2017)).

FIG. 2 depicts exemplary data of PAX3-FOXO1 function with respect to the pGreenFire green fluorescent protein (GFP)-luciferase reporter sequence (SEQ ID NO: 1) used for the high throughput screening assay.

FIG. 3 provides exemplary images depicting visualization via the pGreenFire green fluorescent protein (GFP)-luciferase reporter in PAX3-FOXO1 fusion positive rhabdomyosarcoma cells but not in RD fusion negative rhabdomyosarcoma cells.

FIG. 4 is a visual representation of the screening strategy employed to identify compounds that disrupt the function of the PAX3-FOXO1 fusion transcription factor in RH4 fusion positive rhabdomyosarcoma cells (see Gryder et al., Nat. Commun. 10: 3004 (2019)).

FIG. 5 depicts a box plot of weighted averages obtained from the initial high throughput screening assay. As can be seen in the Figure, of 62,643 tested compounds, 573 were identified as hits by the initial screen.

FIG. 6 is a visual depiction of the high throughput screening assay. As can be seen in the Figure, the follow up screen identified 64 compounds with selective activity.

FIG. 7 depicts an exemplary dose response curve for compound PFI-63 obtained during a dose response follow-up screen of the initial hits.

FIG. 8 is a diagram depicting the effect of PFI-63 on the cell number as measured by confluency of the RH4 and RH30 fusion positive rhabdomyosarcoma cell lines.

FIG. 9 is a diagram depicting the effect of PFI-63 on the cell number as measured by confluency of the RH30.19luc fusion positive rhabdomyosarcoma cell lines.

FIG. 10 depicts an exemplary dose response curve for PFI-63 in RH4 cells at the 72 hour time point. The observed IC₅₀ is 3660 nM.

FIG. 11 depicts an exemplary dose response curve for PFI-63 in RH30 cells at the 72 hour time point. The observed IC₅₀ is 4815 nM.

FIG. 12 depicts the results of a gene set enrichment assay (GSEA; see Subramanian et al., Proc. Natl. Acad. Sci. USA, 102: 15545-15550 (2005)) investigating the cellular effects of PFI-63. The results indicate that targets of the PAX3-FOXO1 fusion transcription factor are downregulated in RH4 cells in the presence of PFI-63. For information on the gene set used for comparison, see Ebauer et al., Oncogene, 26: 7267-7281 (2007).

FIG. 13 depicts the results of a gene set enrichment assay (GSEA) investigating the cellular effects of PFI-63. The results indicate that targets of the PAX3-FOXO1 fusion transcription factor are downregulated in RH4 cells in the presence of PFI-63. For information on the gene set used for comparison, see Davicioni et al., Cancer Res., 66: 6936-6946 (2006).

FIG. 14 depicts the results of a gene set enrichment assay (GSEA) investigating the cellular effects of PFI-63. The results indicate that targets of the PAX3-FOXO1 fusion transcription factor are downregulated in RH4 cells in the presence of PFI-63. For information on the gene set used for comparison, see Cao et al., Cancer Res., 70: 6497-6508 (2010).

FIG. 15 depicts the results of a gene set enrichment assay (GSEA) investigating the cellular effects of PFI-63. The results indicate that targets of the PAX3-FOXO1 fusion transcription factor are downregulated in RH4 cells in the presence of PFI-63. For information on the gene set used for comparison, see Gryder et al., Cancer Discov., 7: 884-899 (2017).

FIG. 16 depicts the results of a gene set enrichment assay (GSEA) investigating the cellular effects of PFI-63. The results indicate that genes that are induced upon KDM3B suppression are upregulated in RH4 cells treated with PFI-63. For information on the gene set used for comparison, see Kuroki et al., Stem Cell Reports, 10: 1340-1354 (2018).

FIG. 17 depicts the results of a gene set enrichment assay (GSEA) investigating the cellular effects of PFI-63. The results indicate that genes that are induced upon KDM3B suppression are upregulated in RH4 cells treated with PFI-63. For information on the gene set used for comparison, see An et al., Biochem. Biophys. Res. Commun., 508: 576-582 (2019).

FIG. 18 depicts the results of a gene set enrichment assay (GSEA) investigating the cellular effects of PFI-63. The results indicate that RH4 cells treated with PFI-63 also experience upregulation of targets associated with myogenic differentiation. For information on the gene set used for comparison, see Liberzon et al., Cell Systems, 1: 417-425 (2015).

FIG. 19 depicts the results of a gene set enrichment assay (GSEA) investigating the cellular effects of PFI-63. The results indicate that RH4 cells treated with PFI-63 also experience upregulation of targets associated with apoptosis. For information on the gene set used for comparison, see Liberzon et al. Cell Systems, 1: 417-425 (2015).

FIG. 20 is an image of the results obtained from a Western blot indicating that treatment with PFI-63 is associated with an increase in MYOG (a transcriptional activator that plays a role in muscle differentiation) in RH4 cells.

FIG. 21 is an image of the results obtained from a Western blot indicating that cleaved PARP (associated with apoptosis) increases in the presence of PFI-63 in RH4 cells.

FIG. 22 depicts the results of a CRISPR/Cas9 genome-scale loss of function time course screen in RH30.19luc cells demonstrating the gene sets that are negatively enriched in the presence of PFI-63 compared to DMSO (no drug). This indicates that inactivation of the genes contained within the gene sets by the single guide RNAs causes selective depletion of the cells from the pool of cells in the presence of PFI-63; i.e., the genes (including targets of histone lysine demethylases) were synthetically lethal with PFI-63.

FIG. 23 depicts the results of a gene set enrichment assay (GSEA) investigating the cellular effects of PFI-63. The results indicate that RH4 cells treated with PFI-63 experience upregulation of targets that are also upregulated by JIB-04, a known multi-histone lysine demethylase inhibitor. For information on the gene set used for comparison, see Wang et al., Nat. Commun., 4: 2035 (2013).

FIG. 24 depicts the results of a gene set enrichment assay (GSEA) investigating the cellular effects of PFI-63. The results indicate that RH4 cells treated with PFI-63 experience upregulation of targets that are also upregulated by GSK-J4, another known multi-histone lysine demethylase inhibitor. For information on the gene set used for comparison, see Hookway, E., The Role of the Lysine Demethylases KDM5 and KDM6 in Bone Malignancies, Ph.D. Thesis, University of Oxford, 2016.

FIG. 25 is an image depicting the results obtained from a Western blot showing a global increase in H3K27me3 (a repressive epigenetic modification to lysine 27 of the histone H3 protein), H3K4me3 (an activating epigenetic modification to lysine 4 of the histone H3 protein), and H3K9me3 (a repressive epigenetic modification to lysine 9 of the histone H3 protein) in cells treated with PFI-63.

FIG. 26 is a diagram illustrating the activating and repressive methylation sites on Histone H3.

FIG. 27 depicts the results of computer based predictions identifying numerous proteins, including several KDMs as potential targets of PFI-63.

FIG. 28 is a computer generated image depicting the predicted docking configuration of PFI-63 in the catalytic domain of KDM3B. For information on the crystal structure of the catalytic domain of KDM3B/JMJD1B used for the simulations, see Vollmar et al., DOI: 10.2210/pdb4C8D/pdb (initial deposition on Sep. 30, 2013, with latest revision on Jan. 24, 2018).

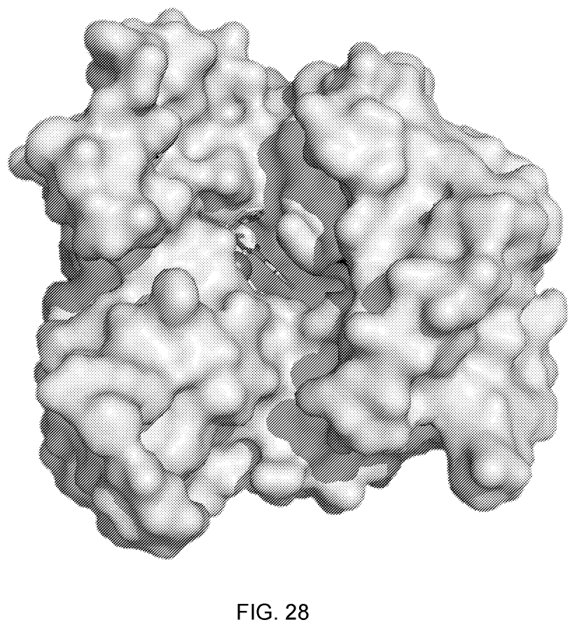

FIG. 29 is a computer generated image depicting the predicted docking configuration of PFI-63 in the catalytic domain of KDM3B.

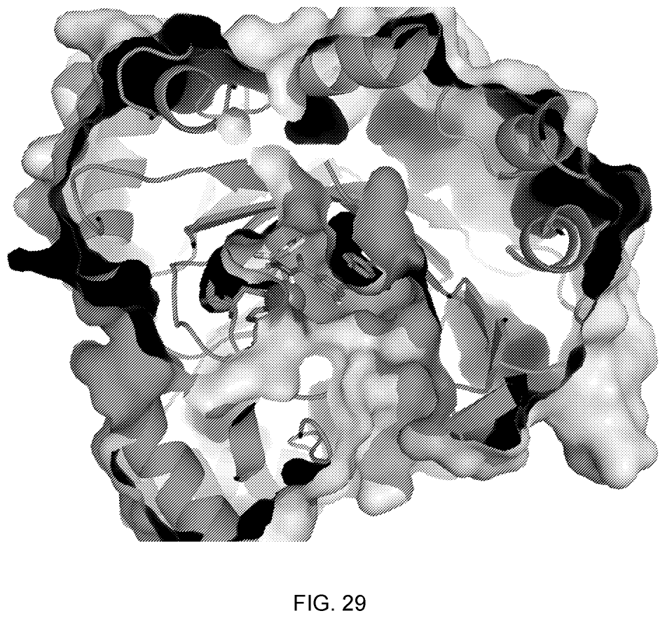

FIG. 30 is a computer generated image depicting the predicted docking configuration of PFI-63 in the catalytic domain of KDM3B.

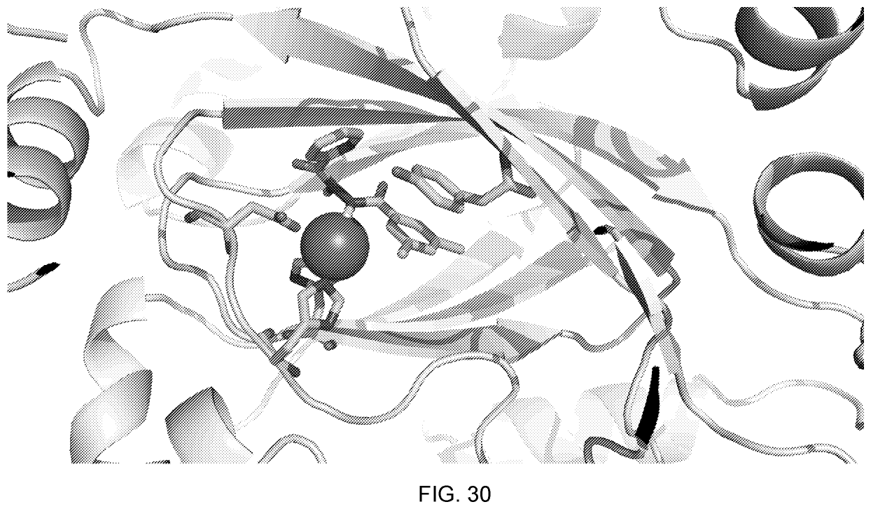

FIG. 31 is a computer generated image depicting an overlay of the catalytic domains of various histone lysine demethylases illustrating the similarities in structure (see Hoffmann et al., Mol. Oncol., 6: 683-703 (2012)).

FIG. 32 depicts an exemplary dose response curve for PFI-90 in RH4 cells at the 72 hour time point. The observed IC₅₀ is 812 nM.

FIG. 33 depicts an exemplary dose response curve for PFI-90 in RH30 cells at the 72 hour time point. The observed IC₅₀ is 3200 nM.

FIG. 34 depicts the results of a gene set enrichment assay (GSEA) investigating the cellular effects of PFI-90. The results indicate that targets of the PAX3-FOXO1 fusion transcription factor are downregulated in RH4 cells in the presence of PFI-90. For information on the gene set used for comparison, see Davicioni et al., Cancer Res., 66:6936-6946, 2006.

FIG. 35 depicts the results of a Gene Set Enrichment Assay investigating the cellular effects of PFI-90. The results indicate that targets of the PAX3-FOXO1 fusion transcription factor are downregulated in RH4 cells in the presence of PFI-90. For information on the gene set used for comparison, see Cao et al., Cancer Res., 70: 6497-6508 (2010).

FIG. 36 depicts the results of a gene set enrichment assay (GSEA) investigating the cellular effects of PFI-90. The results indicate that targets of the PAX3-FOXO1 fusion transcription factor are downregulated in RH4 cells in the presence of PFI-90. For information on the gene set used for comparison, see Gryder et al., Cancer Discov., 7: 884-899 (2017).

FIG. 37 depicts the results of a gene set enrichment assay (GSEA) investigating the cellular effects of PFI-90. The results indicate that genes that are induced upon KDM3B suppression are upregulated in RH4 cells treated with PFI-90. For information on the gene set used for comparison, see Kuroki et al., Stem Cell Reports, 10: 1340-1354 (2018).

FIG. 38 depicts the results of a gene set enrichment assay (GSEA) investigating the cellular effects of PFI-90. The results indicate that genes that are induced upon KDM3B suppression are upregulated in RH4 cells treated with PFI-90. For information on the gene set used for comparison, see An et al., Biochem. Biophys. Res. Commun., 508: 576-582 (2019).

FIG. 39 depicts the results of a gene set enrichment assay (GSEA) investigating the cellular effects of PFI-90. The results indicate that RH4 cells treated with PFI-90 also experience upregulation of targets associated with myogenic differentiation. For information on the gene set used for comparison, see Liberzon et al. Cell Systems, 1: 417-425 (2015).

FIG. 40 depicts the results of a gene set enrichment assay (GSEA) investigating the cellular effects of PFI-90. The results indicate that RH4 cells treated with PFI-90 also experience upregulation of targets associated with apoptosis. For information on the gene set used for comparison, see Liberzon et al. Cell Systems, 1: 417-425 (2015).

FIG. 41 is an image of the results obtained from a Western blot indicating that as the concentration of PFI-90 increases, the methylation of histone H3 also increases.

FIG. 42 is a graphical depiction of the Western blot results indicating that as the concentration of PFI-90 increases, the methylation of histone H3 also increases.

FIG. 43 is a graphical depiction of the results of an investigation into whether PFI-63 induces apoptosis in RH4 and SCMC fusion positive rhabdomyosarcoma cells. As can be seen in the Figure, in both RH4 and SCMC cells, treatment with PFI-63 leads to increased caspase 3, which is associated with apoptosis.

FIG. 44 is a graphical depiction of the results of an investigation into whether PFI-90 induces apoptosis in RH4 and SCMC cells. As can be seen in the Figure, in both RH4 and SCMC cells, treatment with PFI-90 leads to increased caspase 3, which is associated with apoptosis.

FIG. 45 is a graphical depiction of the results of an investigation into whether PFI-90 and PFI-63 induce apoptosis in RH4 cells via annexin V staining. FIG. 45 depicts the results obtained for the DMSO control.

FIG. 46 is a graphical depiction of the results of an investigation into whether PFI-90 and PFI-63 induce apoptosis in RH4 cells via annexin V staining. FIG. 46 depicts the results obtained for PFI-90, in which an increase in cell death (relative to DMSO control) is observed.

FIG. 47 is a graphical depiction of the results of an investigation into whether PFI-90 and PFI-63 induce apoptosis in RH4 cells via annexin V staining. FIG. 47 depicts the results obtained for PFI-63, in which an increase in cell death (relative to DMSO control) is observed.

FIG. 48 is an image depicting the results obtained from a Western blot that indicates that in RH4 cells treated with 3 μM PFI-90, cleaved PARP (associated with apoptosis) increases at 24 hours.

FIG. 49 is an image depicting the results obtained from a Western blot that indicates that in RH4 cells treated with 3 μM PFI-90, PAX3-FOXO1 decreases at 24 hours. This coincides with the increase of cleaved PARP depicted in FIG. 48.

FIG. 50 is a graphical depiction of the results of screening PFI-90 against various leukemia cell lines in the NCI-60 panel. The results indicate that PFI-90 inhibits cell growth.

FIG. 51 is a graphical depiction of the results of screening PFI-90 against various non-small cell lung cancer cell lines in the NCI-60 panel. The results indicate that PFI-90 inhibits cell growth.

FIG. 52 is a graphical depiction of the results of screening PFI-90 against various CNS cancer cell lines in the NCI-60 panel. The results indicate that PFI-90 inhibits cell growth.

FIG. 53 is a graphical depiction of the results of screening PFI-90 against various melanoma cell lines in the NCI-60 panel. The results indicate that PFI-90 inhibits cell growth.

FIG. 54 is a graphical depiction of the results of screening PFI-90 against various prostate cancer cell lines in the NCI-60 panel. The results indicate that PFI-90 inhibits cell growth.

FIG. 55 is a graphical depiction of the results of screening PFI-90 against various colon cancer cell lines in the NCI-60 panel. The results indicate that PFI-90 inhibits cell growth.

FIG. 56 is a graphical depiction of the results of screening PFI-90 against various ovarian cancer cell lines in the NCI-60 panel. The results indicate that PFI-90 inhibits cell growth.

FIG. 57 is a graphical depiction of the results of screening PFI-90 against various breast cancer cell lines in the NCI-60 panel. The results indicate that PFI-90 inhibits cell growth.

FIG. 58 is a graphical depiction of the results of screening PFI-90 against various renal cancer cell lines in the NCI-60 panel. The results indicate that PFI-90 inhibits cell growth and induces cell death.

FIG. 59 depicts an exemplary dose response curve for PFI-63 in OSA-CL osteosarcoma cells at the 72 hour time point. The observed IC₅₀ is 7570 nM.

FIG. 60 depicts an exemplary dose response curve for PFI-90 in OSA-CL cells at the 72 hour time point. The observed IC₅₀ is 1895 nM.

FIG. 61 depicts an exemplary dose response curve for PFI-63 in TC-32 Ewing's sarcoma cells at the 72 hour time point. The observed IC₅₀ is 1880 nM.

FIG. 62 depicts an exemplary dose response curve for PFI-90 in TC-32 cells at the 72 hour time point. The observed IC₅₀ is 1113 nM.

FIG. 63 depicts NMR peaks for PFI-90. All peaks are clearly resolved. The buffer used was 50 mM d-Tris, 80 uM ZnSO₄H₂O, at pH 7.5. Samples contained 300 μM PFI-90, 100 μM 2-OG, 300 μM NMV, 10% D20, 5% D6-DMSO.

FIG. 64 depicts NMR peaks obtained for uketoglutarate (also called 2-oxoglutaric acid or “2-OG”). 2-OG peaks are easily resolved. The buffer was 50 mM d-Tris, 80 uM ZnSO₄H₂O, at pH 7.5. Samples contained 300 μM PFI-90, 100 μM 2-OG (where present), 300 μM NMV, 10% D20, 5% D6-DMSO.

FIG. 65 depicts results from an NMR analysis of PFI-90. As can be seen, chemical shift perturbation upon addition of a truncated KDM3B catalytic domain are not influenced by the presence of 2-OG. The buffer used was 50 mM d-Tris, 80 uM ZnSO₄H₂O, at pH 7.5. Samples contained 300 uM PFI-90, 100 uM 2-OG (where present), 300 uM NMV, 10% D20, 5% D6-DMSO and 12 uM truncated KDM3B catalytic domain (where present). Samples were normalized against DMSO.

FIG. 66 depicts results from a WaterLOGSY experiment. Results show that PFI-90 binds the truncated KDM3B catalytic domain at 300 μM in the presence and in the absence of 2-OG. 2-OG does not bind at 100 μM. Samples were normalized against NMV

FIG. 67 depicts results from a CPMG experiment. Strong binding of PFI-90 to the truncated KDM3B catalytic domain is observed in the presence and absence of 2-OG. 2-OG does not bind at 100 μM. Complete/near complete height attenuation is observed in 5 out of 9 PFI-90 peaks, 70% attenuation is observed in 1 of 9 peaks and 50% attenuation is observed in 3 of 9 peaks. Samples normalized against NMV. The NMV reference peak is normalized to 1000. Minimal baseline correction was applied to the CPMG spectra (Whitaker smoother filter=58, smooth factor=16384).

### DETAILED DESCRIPTION OF THE INVENTION

### Histone Demethylase Inhibitors

The inventors have discovered that certain compounds function as histone demethylase inhibitors. One of the compounds is defined by the structure:

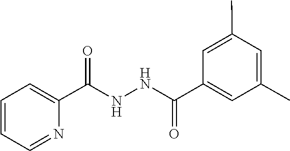 [SMILES: Cc1cc(C)cc(C(=O)NNC(=O)c2ccccn2)c1]

or a pharmaceutically acceptable salt thereof. This compound is referred to herein as “PFI-63” or “UPCMLDOENAT5834780” (Molport Catalog No. MolPort-004-589-395). Another of the compounds is defined by the structure:

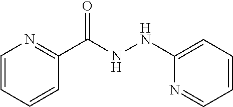 [SMILES: O=C(NNc1ccccn1)c1ccccn1]

or a pharmaceutically acceptable salt thereof. This compound is referred to herein as PFI-90 (Enamine Catalog No. Z1610556569). The inventors have discovered that PFI-63 and PFI-90, as well as pharmaceutically acceptable salts thereof, can be used to treat cancer in mammals by administering one or more of the compounds to a mammal that has cancer and to inhibiting a histone demethylase in cells by contacting the cells with one or more of the compounds.

As used herein, the term “inhibit”, “inhibiting”, or “inhibition” means that a biological response is decreased in the presence of a compound when compared to biological response that occurs in the absence of the compound. For example, in an embodiment, a compound of the invention is administered to a cell such that the biological activity of a histone demethylase in the cell is reduced when compared to the activity of the histone demethylase in the absence of the compound (i.e., the histone demethylase is “inhibited”). The activity of histone demethylases in the presence and absence of a histone demethylase inhibitor may be determined using conventional medical, diagnostic and laboratory techniques known to those skilled in the art.

Those skilled in the art will be familiar with various histone demethylases. In some embodiments, the inhibited histone demethylase may be a lysine specific histone demethylase (also called “histone lysine demethylase” or “KDM”). Various lysine-specific histone demethylases are known in the art. For example, the KDM1 family includes the histone demethylases KDM1A and KDM1B. KDM1A acts on mono- and dimethylated H3K4 and H3K9 (the 4^{th} and 9^{th} lysine residues of histone subunit 3, respectively). KDM1B acts on only mono- and dimethylated H3K4. As another example, the KDM2 family includes KDM2A and KDM2B. KDM2A (also referred to as JHDM1A/FBXL11) can act on mono- and dimethylated H3K36 and trimethylated H3K4. KDM2B (also referred to as JHDM1B/FBXL10) acts only on mono- and dimethylated H3K36. As another example, the KDM3 family includes KDM3A, KDM3B, and JMJD1C. KDM3A (also referred to as JHDM2A/JMJD1A/TSGA) can act on mono- and dimethylated H3K9. KDM3B (also referred to as JHDM2B/JMJD1B) can also act on mono- and dimethylated H3K9 (see Brauchle et al., PLoS ONE, 8: e60549 (2013)). The substrates for JMJD1C (also referred to as JHDM2C/TRIP8) have not been elucidated.

In some embodiments the histone demethylase inhibitors inhibit one or more of KDM3A, KDM3B, KDM5A, KDM5B, KDM4B, and KDM6B. In certain embodiments, the histone demethylase inhibitors inhibit the function of KDM3B. KDM3B is known in the art to epigenetically control colorectal cancer through Wnt signaling (see Li et al., Nat. Commun., 8: 15146 (2017) [DOI: 10.1038/ncomms15146]). KDM3B also transcriptionally activates lmo2 in leukemias (see Kim et al., Mol. Cell. Biol., 32: 2917-2933 (2012)). KDM3B is also upregulated in taxane-platin-chemoresistant non-small cell lung cancer and confers hypersensitivity to histone demethylase inhibitors (Dalvi et al., Cell Reports, 19: 1669-1684 (2017)). KDM3B also regulates the transcriptional network of cell-cycle genes in hepatocarcinoma cells (An et al., Biochem. Biophys. Res. Commun., 508: 576-582 (2019)).

In certain embodiments, the inhibited histone demethylases are associated with the function of an oncogene, e.g., an oncogenic transcription factor. For example, in certain embodiments, the inhibited histone demethylases are associated with the function of oncogenic PAX3/7-FOXO1 fusion transcription factors in fusion positive rhabdomyosarcoma. PAX3/7-FOXO1 fusion transcription factors are the result of chromosomal translocations and are associated with the majority of cases of alveolar rhabdomyosarcoma. In some embodiments, the histone demethylase inhibitors of the present invention disrupt the oncogenic activity of PAX3/7-FOXO1. In other embodiments, the inhibited histone demethylase(s) are associated with the function of the oncogenic EWS/FLI fusion transcription factor, which may be present in Ewing's sarcoma. In some embodiments, the histone demethylase inhibitors of the invention disrupt the oncogenic activity of EWS/FLI.

The histone demethylase inhibitor may target multiple histone demethylases. For example multiple KDMs may be inhibited. Those skilled in the art will recognize such activity as desirable. For example, by inhibiting multiple KDMs in rhabdomyosarcoma cells, a compound may upregulate myogenesis and apoptosis, while at the same time down regulating PAX3-FOXO1 oncogenic targets. As another example, the ability to target multiple KDMs may reduce the frequency of acquired drug resistance arising as a result of acquisition of mutations in a single therapeutic KDM target.

### Methods of Treatment

The invention further provides a method of treating cancer in a mammal comprising, consisting essentially of or consisting of administering to the mammal a therapeutically effective amount of one or both of the compounds described herein or pharmaceutically acceptable salts thereof.

The invention also provides any of the compounds described herein or a pharmaceutically acceptable salt thereof for use in treating cancer in a mammal. The invention further provides the use of a compound or a pharmaceutically acceptable salt thereof in the manufacture of a medicament for treating cancer in a mammal, wherein the compound is any of the compounds described herein or a pharmaceutically acceptable salt thereof. The medicament typically is a pharmaceutical composition as described herein.

The term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). Mammals include non-human primates, e.g., of the order Primates, Ceboids, or Simoids (monkeys) or of the order Anthropoids (humans and apes). In some embodiments, the mammal may be a mammal of the order Rodentia, such as mice and hamsters. In still other embodiments, the mammal is a non-human primate or a human. In some embodiments, the mammal is a human. In some embodiments, the mammal is a human infant, child or adolescent.

Cancers include, without limitation, leukemias (e.g., acute leukemia, acute lymphocytic leukemia, acute myelocytic (myeloid) leukemia, acute myeloblastic leukemia, acute promyelocytic leukemia, acute myelomonocytic leukemia, acute monocytic leukemia, acute erythroleukemia, chronic leukemia, chronic myelocytic (myeloid) leukemia, and chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, (malignant) mesothelioma, Ewing's sarcoma, leiomyosarcoma, rhabdomyosarcoma (including alveolar), colon carcinoma (colon cancer), pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct/intrahepatic bile duct carcinoma, choriocarcinoma, seminoma, embryonal carcinoma, Wilm's tumor, cervical cancer, uterine cancer, testicular cancer, lung carcinoma (lung cancer), small cell lung carcinoma, bladder carcinoma (urinary bladder cancer), epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma). The cancers may include bone cancer, brain cancer, cancer of the anus, anal canal, or anorectum, cancer of the eye, cancer of the joints, cancer of the neck, gallbladder, or pleura, cancer of the nose, nasal cavity, or middle ear, cancer of the oral cavity, cancer of the vulva, esophageal cancer, gastrointestinal carcinoid tumor, hypopharynx cancer, kidney cancer, larynx cancer, liver cancer, nasopharynx cancer, non-small cell lung cancer, peritoneum, omentum, and mesentery cancer, pharynx cancer, rectal cancer, renal cancer, small intestine cancer, soft tissue cancer, stomach cancer, thyroid cancer, and ureter cancer.

The cancer to be treated according to the invention may be responsive to inhibition of a histone demethylase or may be caused by or associated with the function of one or more histone demethylases. In certain embodiments, the cancer is a pediatric cancer, i.e., a form of cancer that occurs primarily in children.

In certain embodiments, the cancer is a transcription driven cancer. As used herein, the term “transcription driven cancer” refers to a cancer in which the primary oncogenic driver is one or more transcription factors, or a chromatin remodeler that have undergone mutations (e.g., translocations) that disrupt the activity of a gene, either by increasing or reducing activity that leads to epigenetic reprogramming of the cells and oncogenic activity. A variety of transcription driven cancers are known to those skilled in the art. Such cancers include, without limitation, anaplastic large cell lymphomas, acute myeloid leukemias, acute lymphoblastic leukemias, acute promyelocytic leukemias, chronic myelogenous leukemias, rhabdomyosarcomas, angiomatoid fibrous histiocytomas, alveolar soft part sarcomas, Ewing's sarcomas, myxoid liposarcomas, myxoid chondrosarcomas, melanomas, liposarcomas, Synovial sarcomas, endometrial stromal sarcomas, Small round-cell sarcomas, and ependymomas.

In some embodiments, the cancer is a transcription driven cancer selected from the group consisting of fusion positive rhabdomyosarcoma and Ewing's sarcoma. For example, in some embodiments, the cancer can be caused by or associated with the function of the PAX3-FOXO1 fusion transcription factor. Such cancers include, e.g., fusion-positive rhabdomyosarcoma.

In some embodiments, the cancer is selected from the group consisting of rhabdomyosarcoma, Ewing's sarcoma, colorectal cancer, ovarian cancer, prostate cancer, CNS cancer, melanoma, breast cancer, leukemia, non-small cell lung cancer, renal cancer, and osteosarcoma.

The terms “treat” and “prevent,” and the like, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention in a mammal. Furthermore, the treatment or prevention provided by the inventive method can include treatment or prevention of one or more conditions or symptoms of the diseases described herein being treated or prevented. Also, for purposes herein, “prevention” can encompass delaying the onset of the disease, or a symptom or condition thereof

### Pharmaceutical Compositions

The invention provides a composition, preferably a pharmaceutical composition, comprising, consisting essentially of or consisting of a compound or salt of PFI-63 and/or PFI-90 as described above and a pharmaceutically acceptable carrier.

The phrase “pharmaceutically acceptable salt” is intended to include nontoxic salts synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Generally, nonaqueous media such as ether, ethyl acetate, ethanol, isopropanol, or acetonitrile are preferred. Lists of suitable salts are found in Remington's Pharmaceutical Sciences, 18th ed., Mack Publishing Company, Easton, Pa., 1990, p. 1445, and Journal of Pharmaceutical Science, 66: 2-19 (1977).

Suitable bases include inorganic bases such as alkali and alkaline earth metal bases, e.g., those containing metallic cations such as sodium, potassium, magnesium, calcium and the like. Non-limiting examples of suitable bases include sodium hydroxide, potassium hydroxide, sodium carbonate, and potassium carbonate. Suitable acids include inorganic acids such as hydrochloric acid, hydrobromic acid, hydroiodic acid, sulfuric acid, phosphoric acid, and the like, and organic acids such as p-toluenesulfonic, methanesulfonic acid, benzenesulfonic acid, oxalic acid, p-bromophenylsulfonic acid, carbonic acid, succinic acid, citric acid, benzoic acid, acetic acid, maleic acid, tartaric acid, fatty acids, long chain fatty acids, and the like. Preferred pharmaceutically acceptable salts of inventive compounds having an acidic moiety include sodium and potassium salts.

It should be recognized that the particular counterion forming a part of any salt of this invention is usually not of a critical nature, so long as the salt as a whole is pharmacologically acceptable and as long as the counterion does not contribute undesired qualities to the salt as a whole.

The above compounds and salts may form solvates, or exist in a substantially uncomplexed form, such as the anhydrous form. As used herein, the term “solvate” refers to a molecular complex wherein the solvent molecule, such as the crystallizing solvent, is incorporated into the crystal lattice. When the solvent incorporated in the solvate is water, the molecular complex is called a hydrate. Pharmaceutically acceptable solvates include hydrates, alcoholates such as methanolates and ethanolates, acetonitrilates and the like. These compounds can also exist in polymorphic forms.

The invention contemplates embodiments in which a compound having a chiral center is a substantially pure enantiomer thereof, a racemic mixture thereof, or a mixture containing any proportion of the two enantiomers thereof. The invention also contemplates all stereoisomers and diastereoisomers of the compounds described herein.

The invention is further directed to a pharmaceutical composition comprising, consisting essentially of or consisting of a pharmaceutically acceptable carrier and at least one compound or salt described herein.

It is preferred that the pharmaceutically acceptable carrier be one that is chemically inert to the active compound(s) in the composition and one that has no detrimental side effects or toxicity under the conditions of use.

The choice of carrier will be determined in part by the particular compound of the invention chosen, as well as by the particular method used to administer the composition. Accordingly, there is a wide variety of suitable formulations of the pharmaceutical composition of the invention.

### Modes of Administration

One skilled in the art will appreciate that various suitable methods of utilizing a compound and administering it to a mammal for the treatment of cancer, which would be useful in the method of the invention, are available. Although more than one route can be used to administer a particular compound, a particular route can provide a more immediate and more effective reaction than another route. Accordingly, the described methods are merely exemplary and are in no way limiting.

The dose administered to a mammal in accordance with the invention should be sufficient to effect the desired response. Such responses include, e.g., reversal or prevention of the negative or deleterious effects of the cancer for which treatment is desired. One skilled in the art will recognize that dosage will depend upon a variety of factors, including the species, age, condition, and body weight of the mammal, as well as the source of the cancer, the particular type of the cancer, and the extent of cancer in the mammal. The size of the dose will also be determined by the route, timing and frequency of administration as well as the existence, nature, and extent of any adverse side-effects that might accompany the administration of a particular compound and the desired physiological effect. It will be appreciated by one of skill in the art that various conditions or disease states may require prolonged treatment involving multiple administrations.

The pharmaceutical composition can be administered parenterally, e.g., intravenously, subcutaneously, intradermally, intramuscularly or intratumorally. Thus, in some embodiments, the invention provides compositions for parenteral administration that comprise a solution of the inventive compound or salt dissolved or suspended in an acceptable carrier suitable for parenteral administration, including aqueous and non-aqueous isotonic sterile injection solutions.

Overall, the requirements for effective pharmaceutical carriers for parenteral compositions are well known to those of ordinary skill in the art. See, e.g., Banker and Chalmers, eds., Pharmaceutics and Pharmacy Practice, J. B. Lippincott Company, Philadelphia, pp. 238-250 (1982), and Toissel, ASHP Handbook on Injectable Drugs, 4th ed., pp. 622-630 (1986). Such solutions can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound or salt of the invention may be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils useful in parenteral formulations include petroleum, animal, vegetable, or synthetic oils. Specific examples of oils useful in such formulations include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-beta-aminopropionates, and 2-alkyl-imidazoline quaternary ammonium salts, and (e) mixtures thereof.

The parenteral formulations can contain preservatives and buffers. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Formulations suitable for oral administration can consist of (a) liquid solutions, such as a therapeutically effective amount of the inventive compound dissolved in diluents, such as water, saline, or orange juice, (b) capsules, sachets, tablets, lozenges, and troches, each containing a predetermined amount of the active ingredient, as solids or granules, (c) powders, (d) suspensions in an appropriate liquid, and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can comprise the active ingredient in a flavor, usually sucrose and acacia or tragacanth, as well as pastilles comprising, consisting essentially of or consisting of the active ingredient in an inert base, such as gelatin and glycerin, or sucrose and acacia, emulsions, gels, and the like containing, in addition to the active ingredient, such excipients as are known in the art.

Suitable doses and dosage regimens can be determined by conventional range-finding techniques known to those of ordinary skill in the art. Generally, treatment is initiated with smaller dosages that are less than the optimum dose of the compound. Thereafter, the dosage is increased by small increments until the optimum effect under the circumstances is reached. The inventive method typically will involve the administration of about 0.001 to about 300 mg of one or more of the compounds described above per kg body weight of the individual. The administration can involve about 0.001 mg, about 0.01 mg, about 0.1 mg, about 1 mg, about 5 mg, about 10 mg, about 20 mg, about 50 mg, about 100 mg, about 200 mg, or about 300 mg or more of one or more of the compounds described above per kg body weight of the individual. Alternatively, or in addition, the administration can involve about 300 mg, about 200 mg, about 100 mg, about 50 mg, about 20 mg, about 10 mg, about 5 mg, about 1 mg, about 0.1 mg, about 0.01 mg, or about 0.001 mg or less of one or more of the compounds described above per kg body weight of the individual. Thus, the administration can be bounded by any two of the aforementioned endpoints. For example, the administration can be about 0.001 mg to about 200 mg, about 0.001 mg to about 1 mg, about 0.01 mg to about 50 mg, about 0.1 mg to about 20 mg, about 1 mg to about 10 mg, about 1 mg to about 20 mg, about 10 mg to about 50 mg, or any other combination of endpoints, of one or more of the compounds described above per kg body weight of the individual.

### Additional Methods

Embodiments of the current invention also include methods for inhibiting histone demethylase(s) in a cell comprising, consisting essentially of or consisting of contacting the cell with one or more of PCFI-63, PFI-90, and pharmaceutically acceptable salts thereof. Such methods may be carried out in vivo or in vitro.

The cell may be any suitable cell. Such cells include, for example, cells derived from mammalian sources. In some embodiments, the cells are human.

The cell may be derived from any tissue type. For example, such cell types include, without limitation, epithelial, endothelial, smooth-muscle, neural, cardiac, and immune cells. An illustrative list of eukaryotic cell types that can be used includes stem cells; pluripotent stem cells; primary cells; fibroblasts; motile cells, ciliated cells; cancer cells including cervix, ovary, colorectal breast, prostate, bladder, pancreas, kidney, lung, salivary gland, testis, cecum, liver, colon, mammary gland, vulva, stomach, pleura, bladder, brain, bone, bone marrow, lymph, eye, connective tissue, pituitary gland, muscle, heart, spleen, skin, uterus, endometrium cells, and epithelial cells; endothelial cells; blood cells; neural cells; secretory cells including adrenal gland cells; contractile cells including smooth muscle cells and skeletal muscle cells; hepatocytes; adipocytes; lymphocytes; macrophages; T-cells; B-cells; dendritic cells; neurons; chondrocytes; and stem cells including embryonic, fetal, amniotic, adult and induced pluripotent stem cells.

In some embodiments, the cell may be a cancer cell. For example, the cells may be derived from, without limitation, leukemias (e.g., acute leukemia, acute lymphocytic leukemia, acute myelocytic (myeloid) leukemia, acute myeloblastic leukemia, acute promyelocytic leukemia, acute myelomonocytic leukemia, acute monocytic leukemia, acute erythroleukemia, chronic leukemia, chronic myelocytic (myeloid) leukemia, and chronic lymphocytic leukemia), polycythemia vera, lymphoma (e.g., Hodgkin's disease and non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, (malignant) mesothelioma, Ewing's sarcoma, leiomyosarcoma, rhabdomyosarcoma (including alveolar), colon carcinoma (colon cancer), pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct/intrahepatic bile duct carcinoma, choriocarcinoma, seminoma, embryonal carcinoma, Wilm's tumor, cervical cancer, uterine cancer, testicular cancer, lung carcinoma (lung cancer), small cell lung carcinoma, bladder carcinoma (urinary bladder cancer), epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma). The cancers may include bone cancer, brain cancer, cancer of the anus, anal canal, or anorectum, cancer of the eye, cancer of the joints, cancer of the neck, gallbladder, or pleura, cancer of the nose, nasal cavity, or middle ear, cancer of the oral cavity, cancer of the vulva, esophageal cancer, gastrointestinal carcinoid tumor, hypopharynx cancer, kidney cancer, larynx cancer, liver cancer, nasopharynx cancer, non-small cell lung cancer, peritoneum, omentum, and mesentery cancer, pharynx cancer, rectal cancer, renal cancer, small intestine cancer, soft tissue cancer, stomach cancer, thyroid cancer, and ureter cancer. In certain embodiments, the cells are rhabdomyosarcoma cells or Ewing's sarcoma cells.

The cells may be cancer cells derived from cancers responsive to inhibition of a histone demethylase or cancers caused by or associated with the function of one or more histone demethylases. In certain embodiments, the cells are derived from a pediatric cancer, i.e., a form of cancer that occurs primarily in children.

In certain embodiments, the cell is a cancer cell derived from a transcription driven cancer. In some embodiments, the cell is derived from a transcription driven cancer selected from the group consisting of fusion positive rhabdomyosarcoma and Ewing's sarcoma. For example, in some embodiments, the cancer can be caused by or associated with the function of the PAX3-FOXO1 fusion transcription factor. Such cancers include, e.g., fusion-positive rhabdomyosarcoma.

In some embodiments, the cell is a cancer cell derived from a cancer selected from the group consisting of rhabdomyosarcoma, Ewing's sarcoma, colorectal cancer, ovarian cancer, prostate cancer, CNS cancer, melanoma, breast cancer, leukemia, non-small cell lung cancer, renal cancer, and osteosarcoma.

In certain embodiments, the methods are carried out in vitro. In such embodiments, the cells may be obtained, stored and utilized in any suitable matter according to procedures known in the art. In some embodiments, the cells may be harvested from a mammal, such as, for example, harvested from a tumor present in the mammal. In some embodiments, the cells may be obtained via cell culture. Cell cultures suitable for the invention include, without limitation, continuous cell lines (e.g., with an immortal phenotype), primary cell cultures, finite cell lines (e.g., non-transformed cells), and any other cell population maintained in vitro, including oocytes and embryos.

The cell may be contacted with the compounds of the invention via any suitable method. For example, suitable methods may include, without limitation, the delivery of the therapeutic compound directly to the cell, delivery of the compound to the vicinity of the cell, delivery of the compound directly to a tumor, delivery of the compound to the vicinity of a tumor, administration of the compound to a patient that has a tumor, or any combination thereof.

The following are certain aspects of the invention:

1. A pharmaceutical composition comprising, consisting essentially of or consisting of (a) one or more compounds selected from the group consisting of

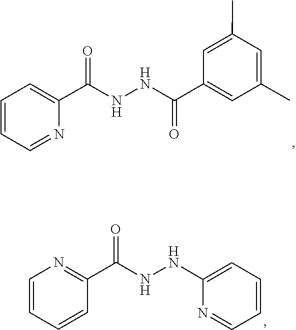 [SMILES: Cc1cc(C)cc(C(=O)NNC(=O)c2ccccn2)c1 ; O=C(NNc1ccccn1)c1ccccn1]

and pharmaceutically acceptable salts thereof and (b) and a pharmaceutically acceptable carrier.

2. The composition of aspect 1, for use in the treatment of cancer.

3. The composition of aspect 2, wherein the cancer is a transcription driven cancer.

4. The composition of aspect 2, wherein the cancer is selected from the group consisting of rhabdomyosarcoma, Ewing's sarcoma, colorectal cancer, ovarian cancer, prostate cancer, CNS cancer, melanoma, breast cancer, leukemia, non-small cell lung cancer, renal cancer, and osteosarcoma.

5. The composition of aspect 2, wherein the cancer is Ewing's sarcoma.

6. The composition of aspect 2, wherein the cancer is rhabdomyosarcoma.

7. The composition of aspect 6, wherein the cancer is alveolar rhabdomyosarcoma.

8. The composition of aspect 6, wherein the cancer is selected from the group consisting of PAX3-FOXO1 fusion transcription factor positive rhabdomyosarcoma and PAX7-FOXO1 fusion transcription factor positive rhabdomyosarcoma.

9. A method of treating cancer in a mammal, comprising, consisting essentially of or consisting of administering an effective amount of one or more compounds selected from the group consisting of:

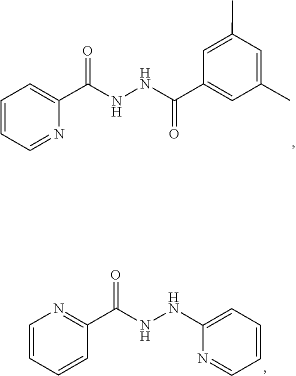 [SMILES: Cc1cc(C)cc(C(=O)NNC(=O)c2ccccn2)c1 ; O=C(NNc1ccccn1)c1ccccn1]

and pharmaceutically acceptable salts thereof, to the mammal.

10. The method of aspect 9, wherein the compound is:

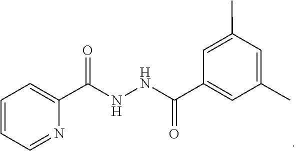 [SMILES: Cc1cc(C)cc(C(=O)NNC(=O)c2ccccn2)c1]

11. The method of aspect 9, wherein the compound is:

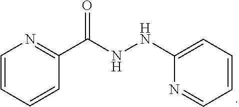 [SMILES: O=C(NNc1ccccn1)c1ccccn1]

12. The method of any one of aspects 9-11, wherein the cancer is a transcription driven cancer.

13. The method of any one of aspects 9-11 wherein the cancer is selected from the group consisting of rhabdomyosarcoma, Ewing's sarcoma, colorectal cancer, ovarian cancer, prostate cancer, CNS cancer, melanoma, breast cancer, leukemia, non-small cell lung cancer, renal cancer, and osteosarcoma.

14. The method of any one of aspects 9-11, wherein the cancer is Ewing's sarcoma.

15. The method of any one of aspects 9-11, wherein the cancer is rhabdomyosarcoma.

16. The method of aspect 15, wherein the cancer is alveolar rhabdomyosarcoma.

17. The method of aspect 15, wherein the cancer is selected from the group consisting of PAX3-FOXO1 fusion transcription factor positive rhabdomyosarcoma and PAX7-FOXO1 fusion transcription factor positive rhabdomyosarcoma.

18. The method of any one of aspects 9-17, wherein a histone demethylase is inhibited by the compound in the mammal.

19. The method of aspect 18, wherein the histone demethylase is a lysine-specific histone demethylase.

20. The method of aspect 19, wherein the lysine specific histone demethylase is selected from the group consisting of KDM3A, KDM3B, KDM5A, KDM5B, KDM4B, and KDM6B.

21. The method of aspect 20, wherein the histone demethylase is KDM3B.

22. The method of aspect 17, wherein the cancer is PAX3-FOXO1 fusion transcription factor positive rhabdomyosarcoma, and wherein the function of the PAX3-FOXO1 fusion transcription factor is disrupted by the compound in the mammal.

23. The method of aspects 17, wherein the cancer is PAX7-FOXO1 fusion transcription factor positive rhabdomyosarcoma, and wherein the function of the PAX7-FOXO1 fusion transcription factor is disrupted by the compound in the mammal.

24. A method for inhibiting a histone demethylase in cells comprising, consisting essentially of or consisting of contacting the cells with one or more compounds selected from the group consisting of:

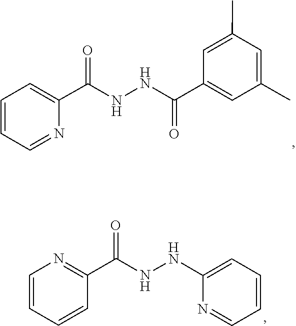 [SMILES: Cc1cc(C)cc(C(=O)NNC(=O)c2ccccn2)c1 ; O=C(NNc1ccccn1)c1ccccn1]

and pharmaceutically acceptable salts thereof.

25. The method of aspect 24, wherein the compound is:

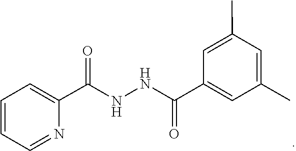 [SMILES: Cc1cc(C)cc(C(=O)NNC(=O)c2ccccn2)c1]

26. The method of aspect 24, wherein the compound is:

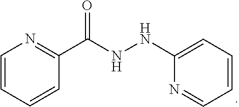 [SMILES: O=C(NNc1ccccn1)c1ccccn1]

27. The method of any one of aspects 24-26, wherein the cells are in vivo.

28. The method of any one of aspects 24-27, wherein the cells are human.

29. The method of any one of aspects 24-28, wherein the cells are cancer cells.

30. The method of aspect 29 wherein the cancer is selected from the group consisting of rhabdomyosarcoma, Ewing's sarcoma, colorectal cancer, ovarian cancer, prostate cancer, CNS cancer, melanoma, breast cancer, leukemia, non-small cell lung cancer, renal cancer, and osteosarcoma.

31. The method of aspect 29, wherein the cancer cells are Ewing's sarcoma cells.

32. The method of aspect 29, wherein the cells are rhabdomyosarcoma cancer cells.

33. The method of aspect 32, wherein the cells are alveolar rhabdomyosarcoma cells.

34. The method of aspect 29, wherein the cancer cells are selected from the group consisting of PAX3-FOXO1 fusion transcription factor positive rhabdomyosarcoma and PAX7-FOXO1 fusion transcription factor positive rhabdomyosarcoma.

35. The method of aspect 34, wherein the cancer is PAX3-FOXO1 fusion transcription factor positive rhabdomyosarcoma, and wherein the function of the PAX3-FOXO1 fusion transcription factor is disrupted by the inhibition of the histone demethylase.

36. The method of aspect 34, wherein the cancer is PAX7-FOXO1 fusion transcription factor positive rhabdomyosarcoma, and wherein the function of the PAX7-FOXO1 fusion transcription factor is disrupted by the inhibition of the histone demethylase.

37. The method of any one of aspects 24-36, wherein the histone demethylase is a lysine specific histone demethylase.

38. The method of aspect 37, wherein the lysine specific histone demethylase is selected from the group consisting of KDM3A, KDM3B, KDM5A, KDM5B, KDM4B, KDM6B.

39. The method of aspect 37, wherein the lysine specific histone demethylase is KDM3B.

### EXAMPLES

The following examples further illustrate the invention but, of course, should not be construed as in any way limiting its scope.

### Example 1

This example describes an assay used to identify compounds that disrupt the function of the PAX3-FOXO1 fusion transcription factor in RH4 fusion-positive rhabdomyosarcoma cells.

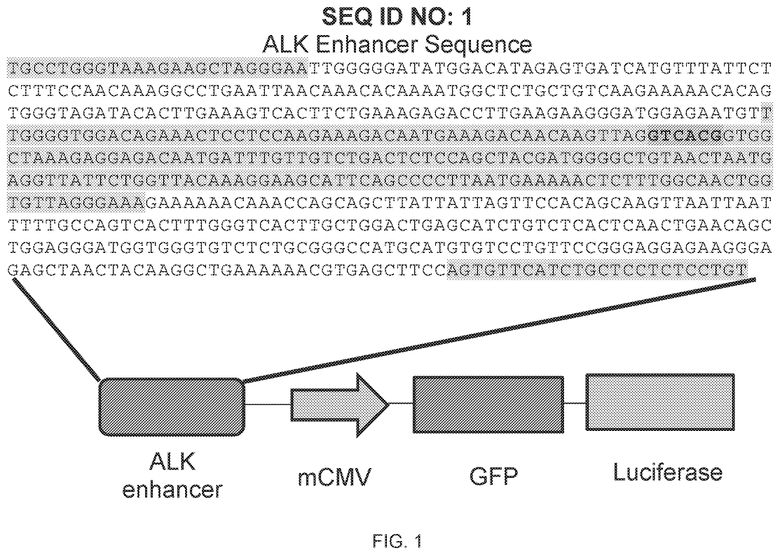

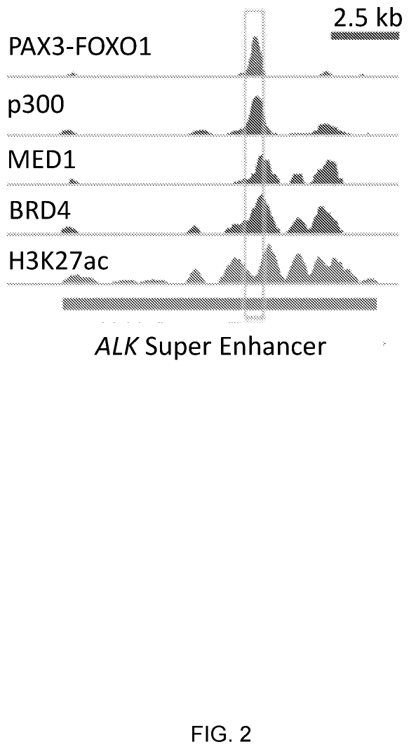

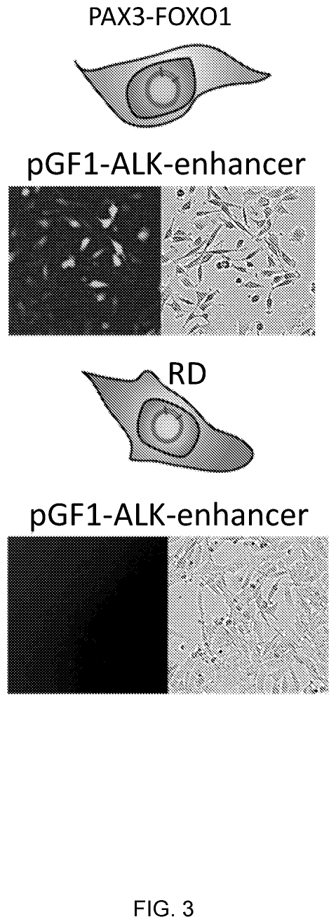

The screen was performed using two derivatives of RH4 FP-RMS cells:
(1) “ALK-Luc cells”—RH4 cells expressing a pGreenFire green fluorescent protein (GFP)-luciferase reporter (SEQ ID NO: 1) controlled by the PAX3-FOXO1-dependent intronic ALK super enhancer (for reporting on PAX3-FOXO1 activity) (FIGS. 1 and 2); and
(2) “CMV-Luc cells”—control RH4 cells in which the reporter is transcribed from a constitutively active cytomegalovirus (CMV) promoter.
   An Example of visualization via the ALK-Luc reporter is depicted in FIG. 3.

The cells were enriched for reporter expression levels and used for assay development. A high throughput screening assay was developed after optimization of cell seeding density, length of incubation of cells prior to and post treatment with test compounds, and effect of passaging among other factors. The screen was designed to identify compounds that suppressed PAX3-FOXO1 activity without disrupting viability (at a short time points) or general transcription. The assay simultaneously monitors the function of the PAX3-FOXO1 super enhancer (also referred to as “ALK super enhancer”), general transcriptional activity as well as cell viability.

The assay was validated for sensitivity and reproducibility over three separate runs using 3000 randomly chosen screening compounds with high correlation (coefficient median R2=0.87, range R2=0.86-0.90).

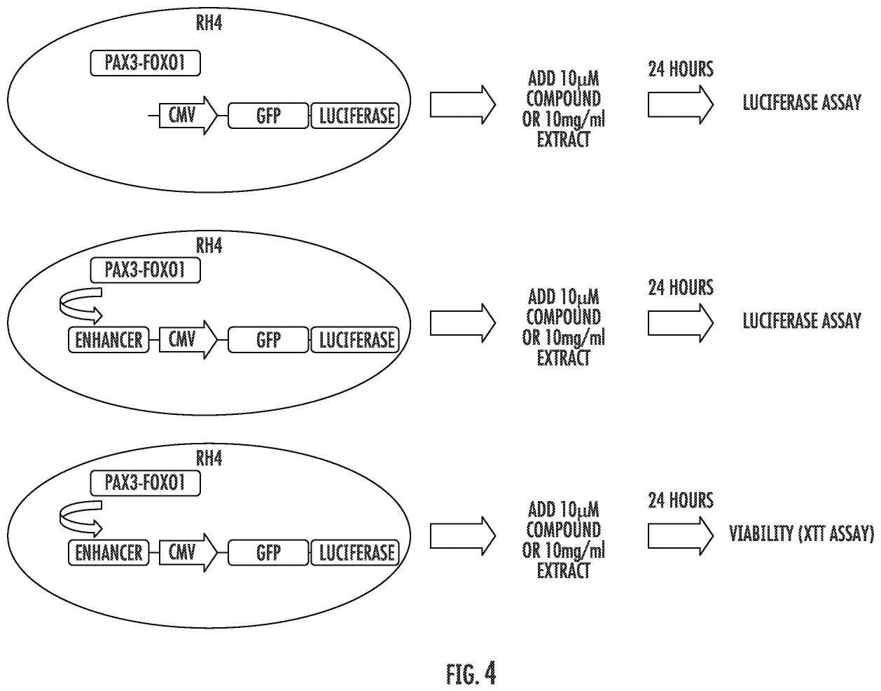

The visual summary of the screening strategy is illustrated in FIG. 4. ALK-Luc cells were seeded in both white-walled and white-bottomed 384-well plates (Perkin Elmer, Cat #6007658) for luciferase assays and in clear 384-well plates (Perkin Elmer, Cat #6007688) for viability assay (XTT reduction) by transferring 27 μL of cell suspensions (seeding 3000 cells per well) into each well. The plates were then transferred into an incubator for 18-20 h. The CMV-Luc cells were similarly seeded into white plates and incubated.

A pure compound library of 62,643 compounds consisting of synthetic and natural products described previously (Grohar et al., J Natl. Cancer Inst., 103: 962-978 (2011)) was then used in a high throughput screening campaign to identify inhibitors of PAX3-FOXO1. Stock DMSO Solutions of these compounds were used to prepare 100 μM solutions in growth medium in 384-well plates also containing 16 wells each of a positive control (Actinomycin D, Sigma, Cat #A1410) and negative control (DMSO). Screening compounds along with the positive and negative controls were added to assay plates by transferring 3 μL of the prepared 100 μM dilutions using an automated liquid handler (Agilent, Bravo). Treated plates were incubated for 24 h. At the end of this period, treated plates were allowed to equilibrate to room temperature for 30 minutes. SteadyLite Plus luciferase assay reagent powder (PerkinElmer, Cat #6066759) was reconstituted in its buffer in parallel and was also allowed to equilibrate to room temperature.

After transferring 30 μL of the luciferase assay reagent using a liquid handler, plates were further incubated at room temperature for 10 minutes. Finally, luminescence measurements were carried out using a multi-label microplate reader (BMG, Pherastar FSX) set in luminescence mode. Cell viability was assessed by adding XTT reagent (10 μL per well) in clear plates containing treated ALK-Luc cells. XTT treated plates were read using a plate reader in absorbance mode at 450 nm (PerkinElmer, Envision) after further incubation for 1 hour to allow cells to metabolize XTT to a colored formazan product. Luminescence and XTT absorbance values were normalized to the average of the negative controls on each plate and were calculated as a percentage of these controls. Performance of the screening assay was routinely monitored via calculation of Z-factor (Zhang et al., J. Biomol. Screen., 4: 67-73 (1999)) with Z′ for all plates found to be >0.7.

A weighted mean of percent-control of the RH4 ALK-Luc (L), RH4 XTT (X), and RH4 CMV-Luc (C) values for each test compound was determined employing the formula:

Weighted average=(4L+2C+X)/7

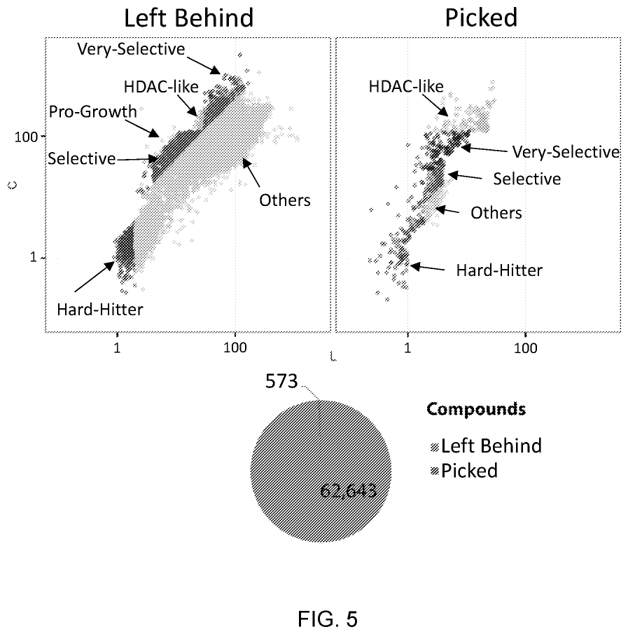

Compounds with weighted averages above 60 were identified as hits (outliers) after preparing a boxplot of weighted averages (see FIG. 5). This resulted in the identification of 573 compounds as hits.

### Example 2

This Example describes hit confirmation and further screening of the identified compounds.

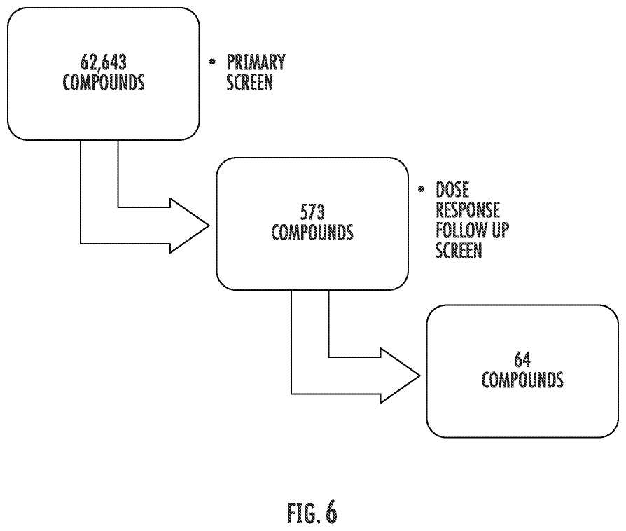

RH4 ALK-Luc and RH4 CMV-Luc cells were seeded into separate white-walled and bottomed 384-well plates and were incubated and treated with screen hits and epigenome compounds. The highest test concentration of these compounds (10-fold) were prepared from DMSO stock solutions via dilution in medium. Final DMSO concentration in assay wells was 0.2% or less. Ten-fold serial dilutions of test compounds were made using an automated liquid handler, and 3 μL of each dilution was added to each well of plates containing RH4 ALK-Luc and RH4 CMV-Luc cells in 27 μL medium. Each test concentration was assayed in quadruplicate. Corresponding DMSO dilutions were used as control. Treated plates were then read following the same procedure used for screening described above. Selective activity was confirmed for 64 compounds (see FIG. 6).

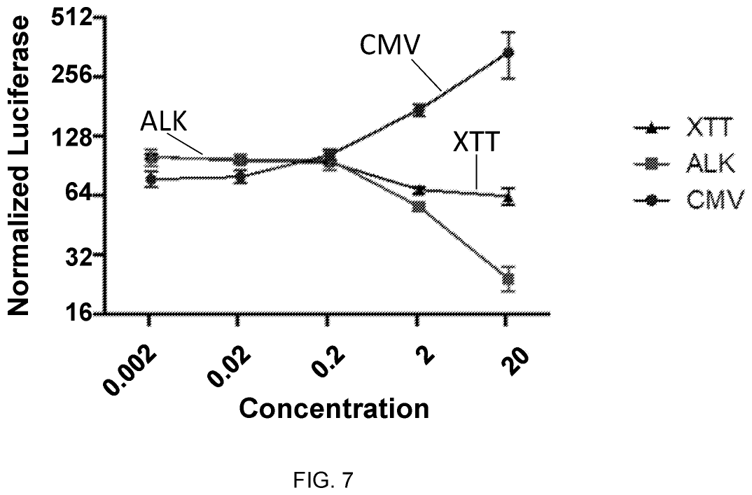

An exemplary dose response curve for PFI-63 is presented as FIG. 7.

### Example 3

This example describes the identification and initial characterization of PFI-63.

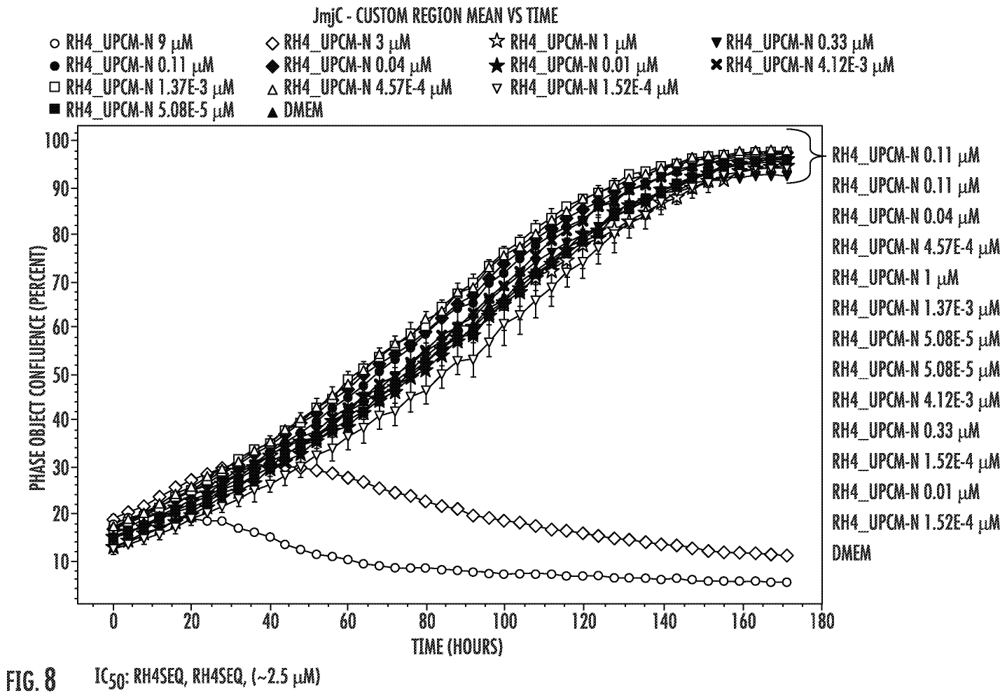

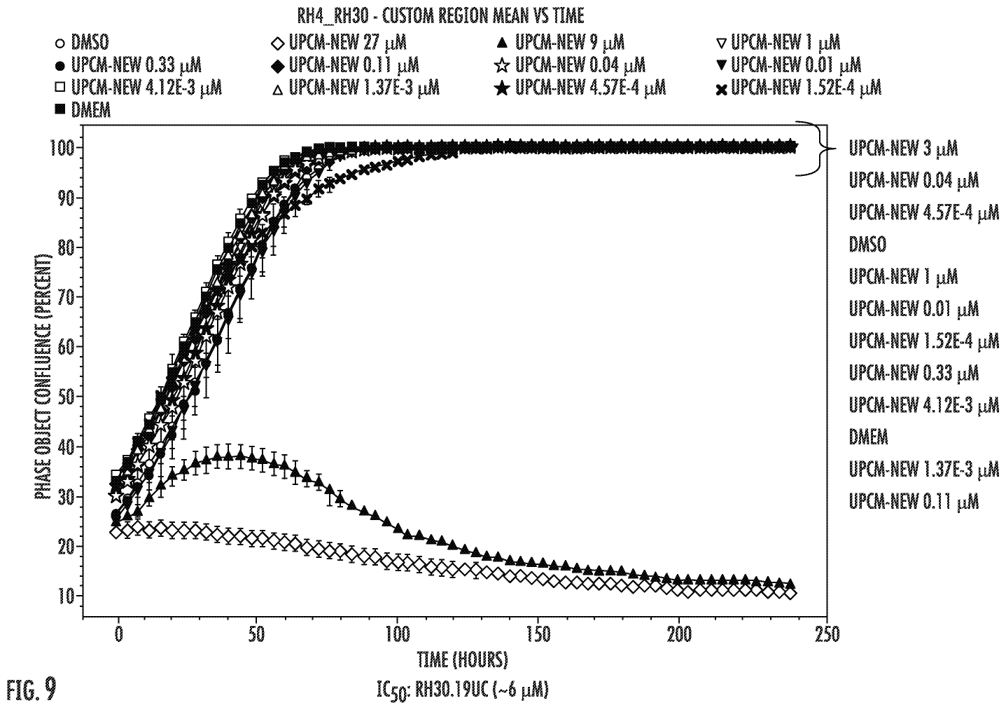

Among the 64 compounds identified above, 33 compounds of unknown mechanism of action were identified. One of them, PFI-63, had significant suppression of PAX3-FOXO1 super enhancer activity, and an IC₅₀ of ˜2-6 μM (FIGS. 8-9). When rhabdomyosarcoma cells are treated with PFI-63 it showed an elongation of cells as confirmed by morphological imaging and apoptosis as measured by cleaved PARP Westerns.

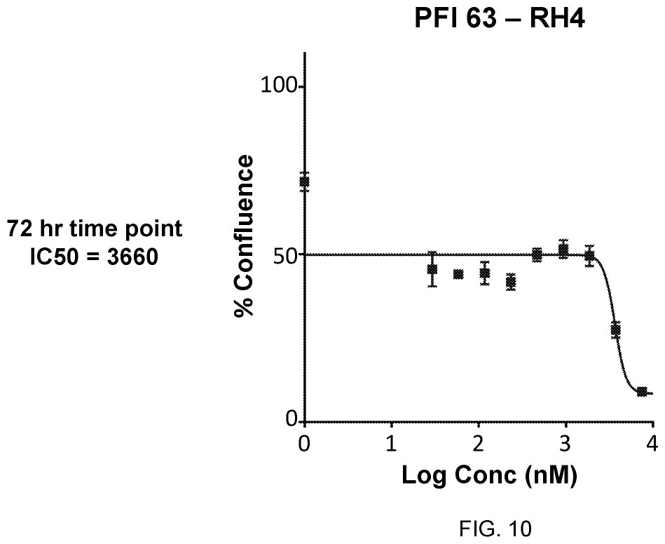

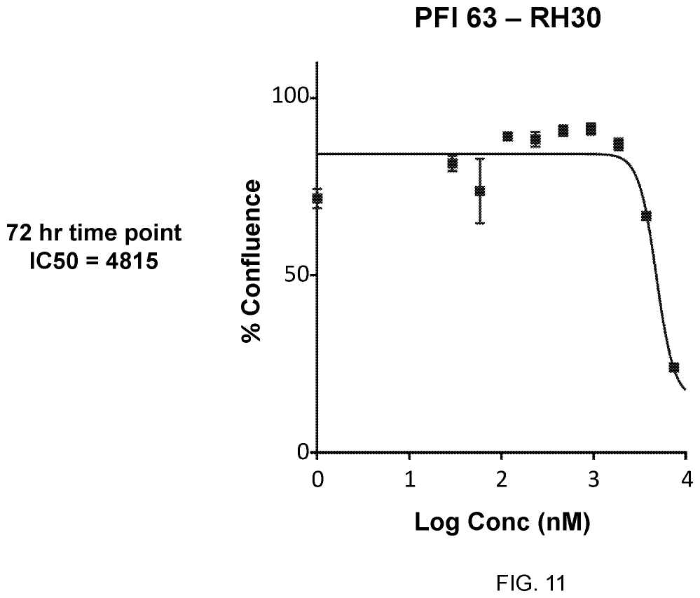

A cell confluence assay was performed to further assess the cytotoxicity of PFI-63 in RH4 and RH30 rhabdomyosarcoma cells. Confluence data was collected via photographic images of cells treated with PFI-63 at the following time points: 24 hours, 48 hours, 72 hours, 96 hours and 120 hours. Exemplary dose response curves at 72 hours are presented as FIGS. 10 and 11 in RH4 and RH30 cells, respectively. The results indicate that PFI-63 is toxic to rhabdomyosarcoma cells (i.e. RH4 and RH30 cells) with an IC₅₀ of approximately 3-5 μM.

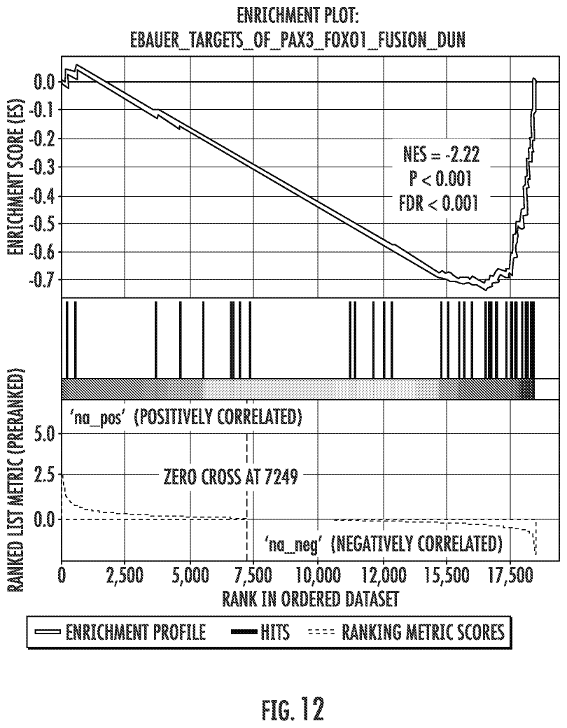

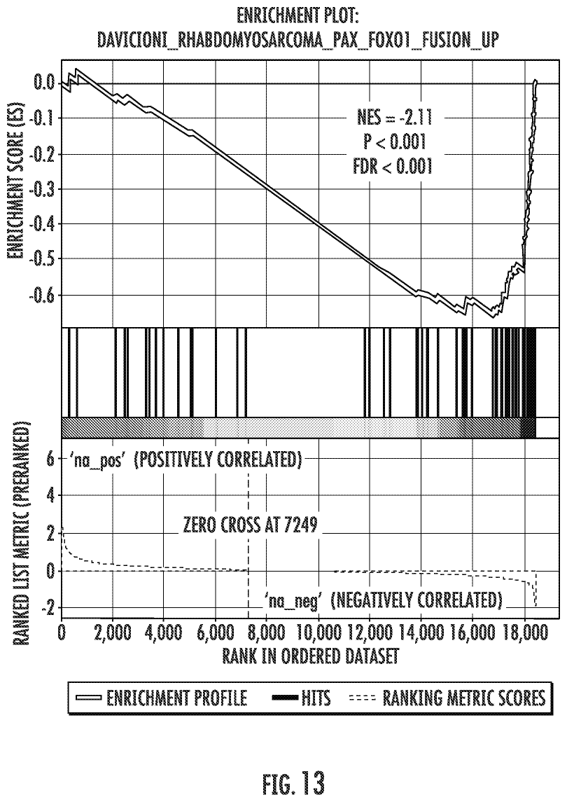

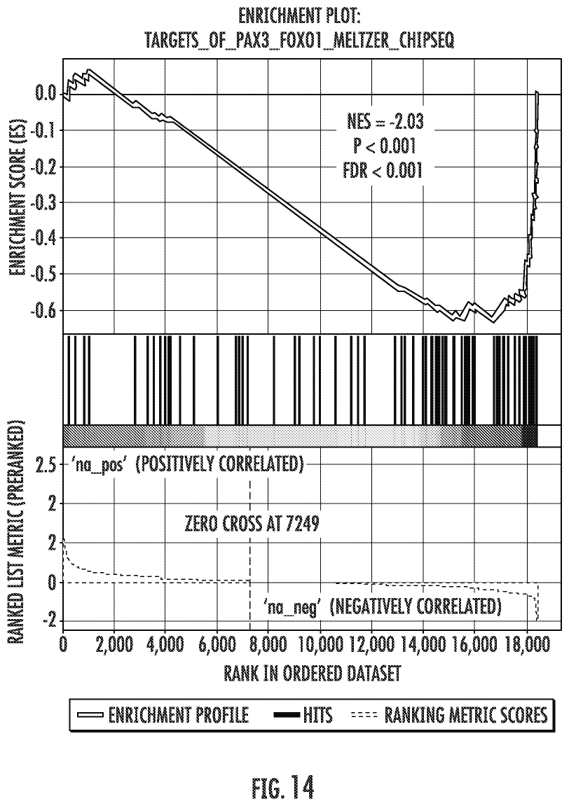

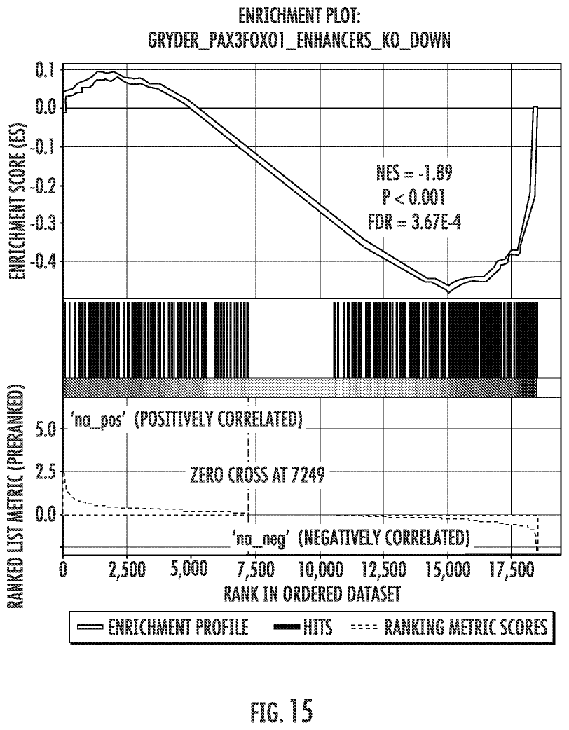

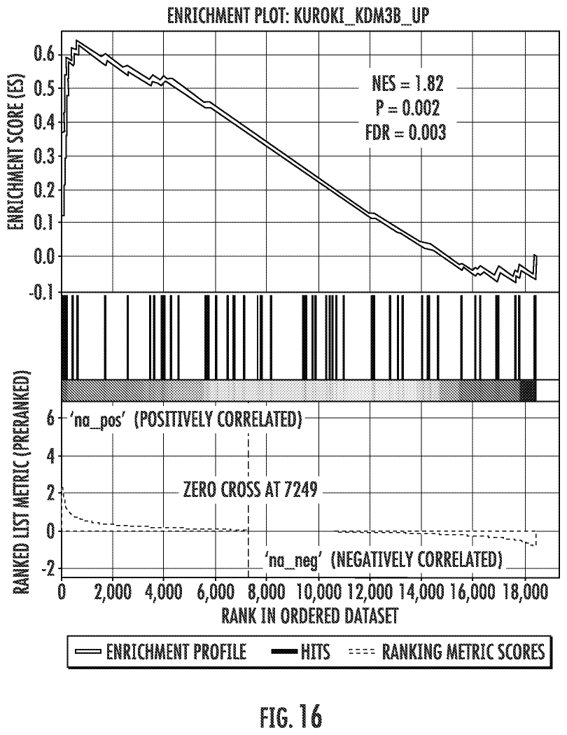

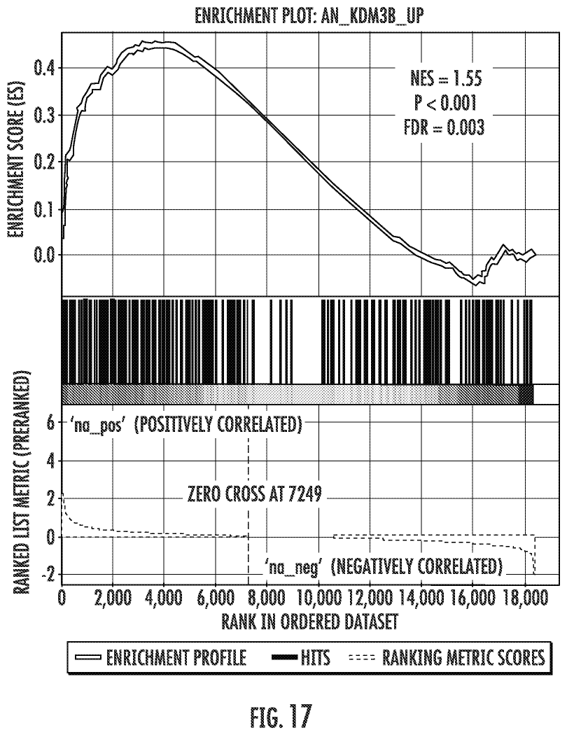

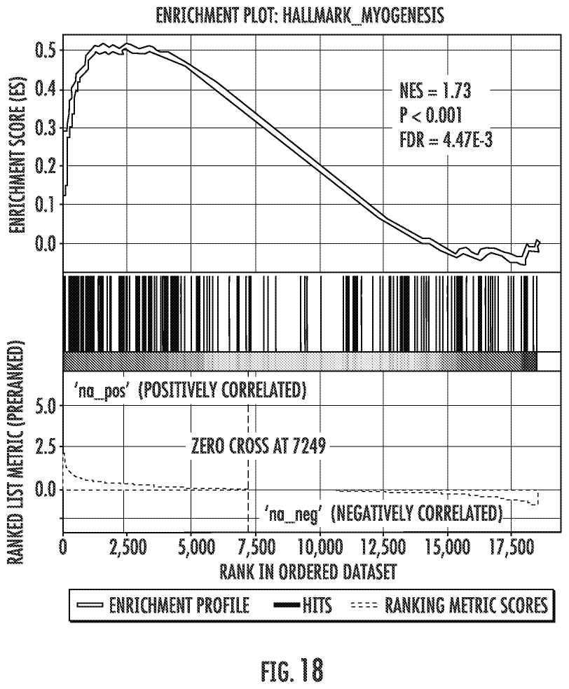

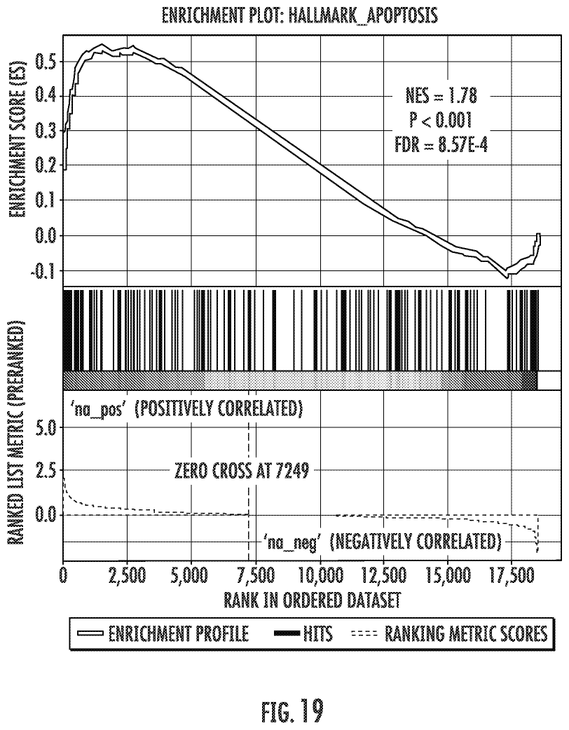

Gene set enrichment analyses (GSEA) were performed according to methods known in the art to evaluate protein expression in cells treated with PFI-63 as compared to control cells. Briefly, gene expression in RH4 cells treated with PFI-63 was assessed by RNASeq, and the results were statistically compared with various pre-existing gene sets. The results indicate that targets of the PAX3-FOXO1 fusion transcription factor are downregulated in the presence of PFI-63 (see FIGS. 12-15). The results also indicate that genes that are induced upon KDM3B suppression are upregulated in RH4 cells treated with PFI-63 (see FIGS. 16 and 17). The results also indicate that RH4 cells treated with PFI-63 experience upregulation of targets associated with myogenic differentiation and apoptosis (see FIGS. 18 and 19).

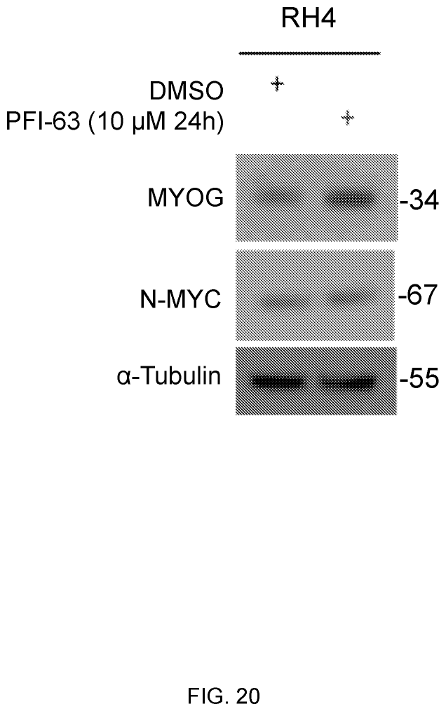

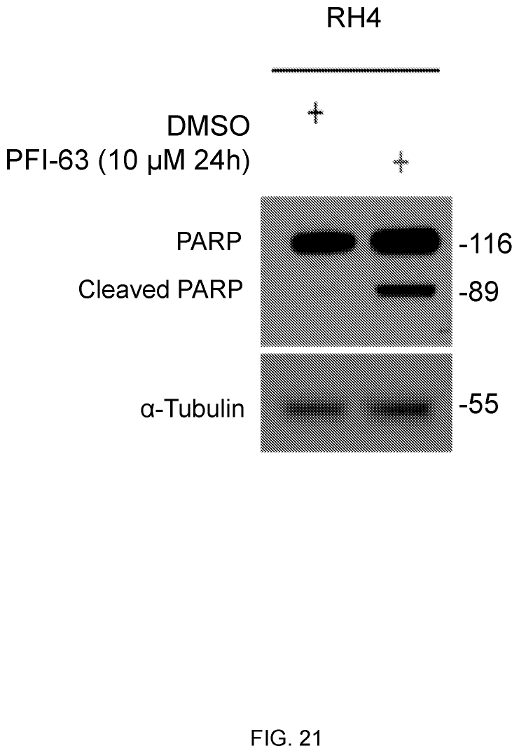

The GSEA results were supported by analysis via Western blot. Western blot analysis indicated that PFI-63 was associated with an increase in MYOG (FIG. 20), which is a transcriptional activator that plays a role in muscle differentiation. Cleaved PARP, associated with apoptosis, also increases in the presence of PFI-63 (FIG. 21). This example confirms that PFI-63 disrupts PAX3-FOXO1 super enhancer circuitry. PFI-63 is also associated with upregulation of targets associated with myogenic differentiation and apoptosis.

### Example 4

This example describes further characterization of PFI-63.

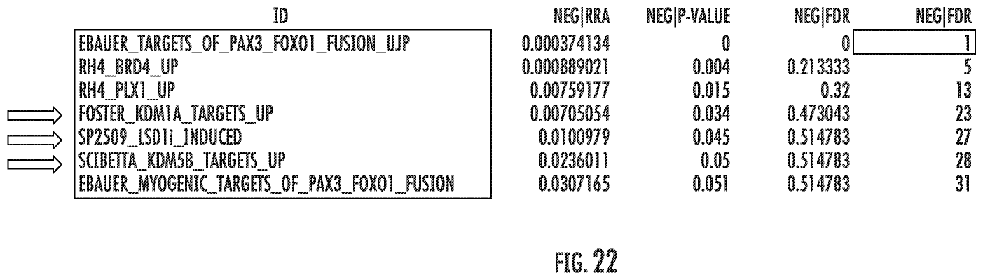

To identify specific proteins targeted by PFI-63, a genome-scale loss of function time course screen was performed in RH30.19luc cells using two CRISPR/Cas9 libraries (GeCKOv2 and TKOv3) with and without treatment with PFI-63. It was hypothesized that the targets or pathways suppressed by the compound and the genes that are required for survival upon treatment with PFI-63 will be synthetically lethal. The CRISPR/Cas9 screen demonstrated that targets of histone lysine demethylases were synthetically lethal with PFI-63 (see FIG. 22).

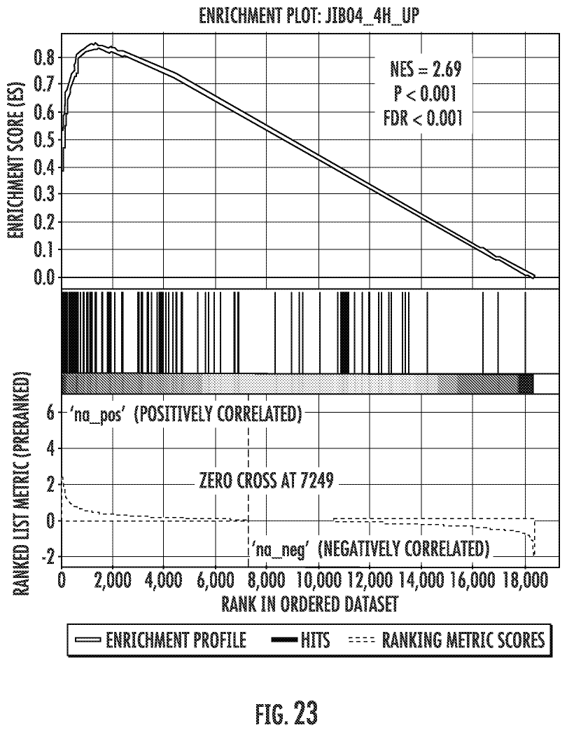

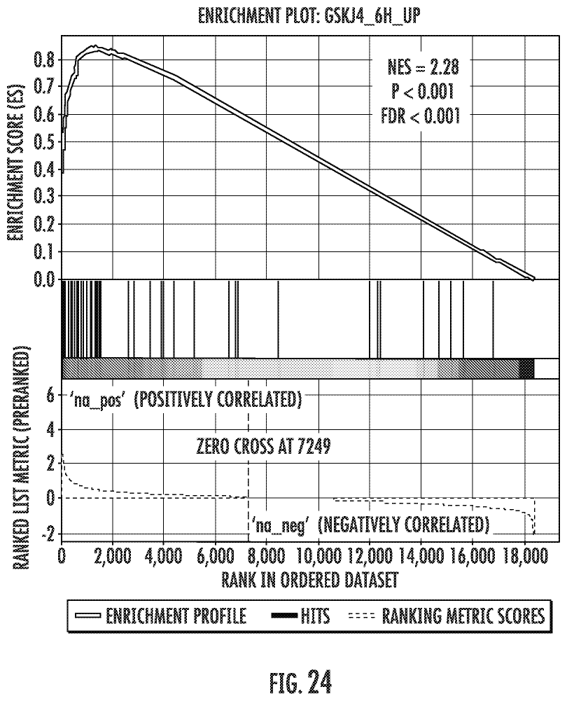

Accordingly, the results from the CRISPR/Cas9 screen suggested comparison of PFI-63 with other active agents with known activity, including JIB-04 and GSK-J4, known multi-histone lysine demethylase inhibitors. The GSEA results demonstrated that targets upregulated by JIB-04 and GSK-J4 are also upregulated in the presence of PFI-63 (see FIGS. 23 and 24). This indicates that PFI-63 is in the same class as JIB-04 and GSK-J4, i.e., a histone lysine demethylase inhibitor.

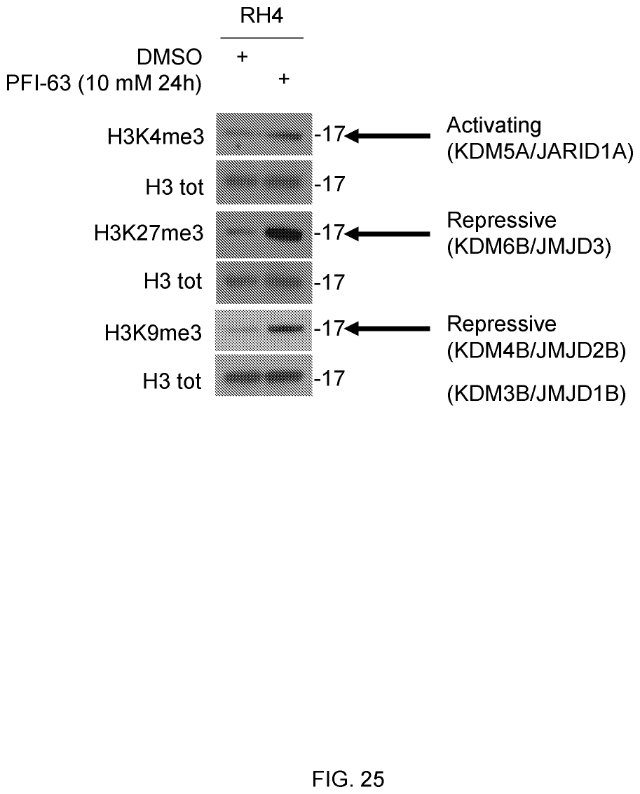

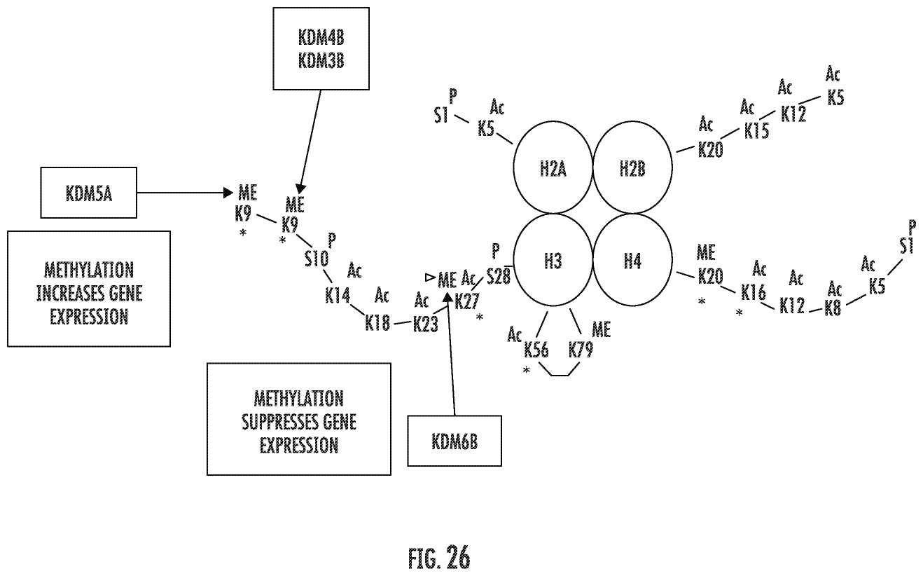

Western blot analysis confirmed that, in cells treated with PFI-63, there is a global increase in H3K27me3 (a repressive epigenetic modification to lysine 27 of the histone H3 protein), H3K4me3 (an activating epigenetic modification to lysine 4 of the histone H3 protein), and H3K9me3 (a repressive epigenetic modification to lysine 9 of the histone H3 protein) (FIG. 25). A diagram illustrating this activity is provided as FIG. 26.

This example further indicates that PFI-63 is a lysine-specific demethylase (KDM) inhibitor.

### Example 5

This example describes software based docking predictions and analysis confirming that PFI-63 is a KDM inhibitor and specifically docks in the catalytic domain of KDM3B.

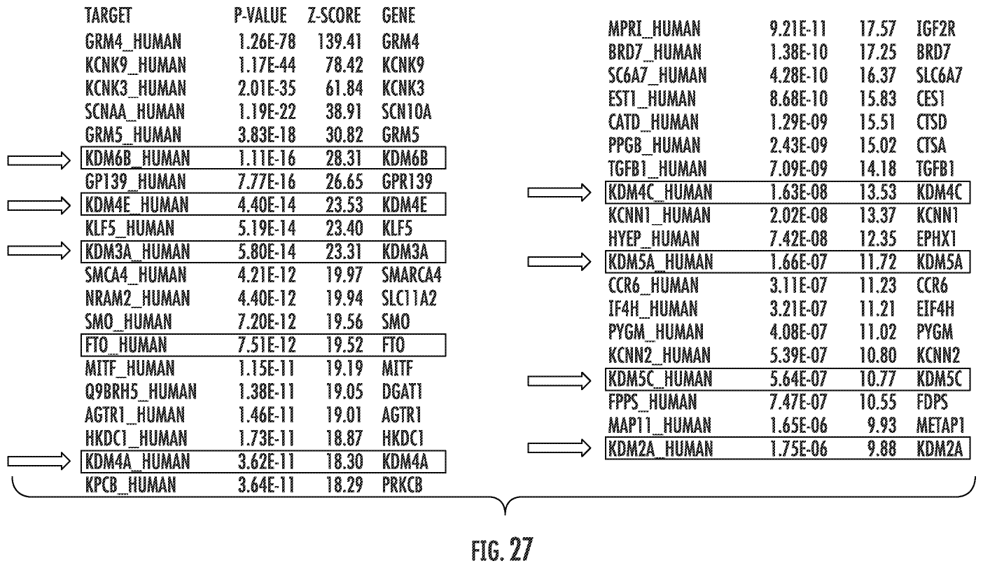

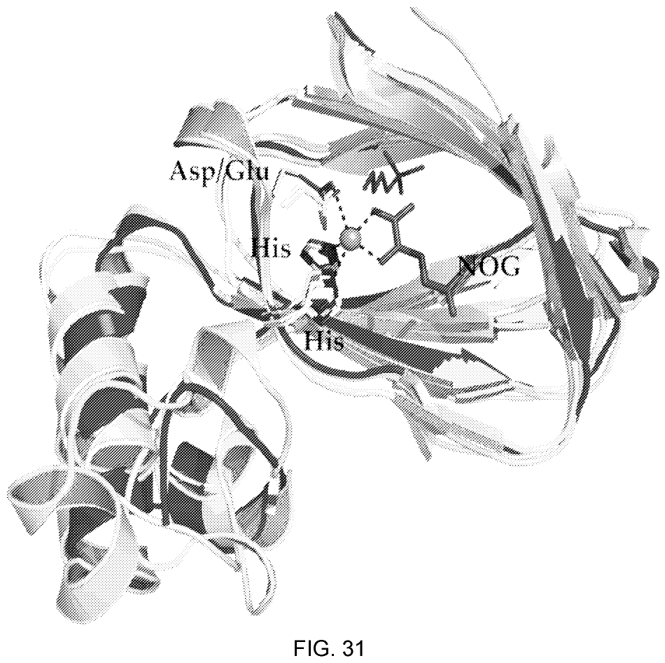

Computer based predictions identified numerous proteins, including several KDMs that are potential targets of PFI-63 (see FIG. 27). Computer simulations confirm that PFI-63 docks in the catalytic domain of KDM3B. Figures, 28, 29 and 30, illustrate the predicted docking configuration at increasing levels of magnification and detail. Those skilled in the art will be aware that that the docking sites of KDMs are quite similar (see FIG. 31), and therefore, these results indicate that PFI-63 may also bind other KDMs.

### Example 6

This example describes PFI-63 inhibitory activity as determined using a histone lysine demethylase panel.

PFI-63 was sent to Eurofins (www.eurofins.com) for direct enzymatic inhibition activity analysis. The enzymatic activity of each of the full length protein was analyzed using known substrates such as methylated lysines. The proteins were treated with various concentrations of PFI-63. The results are presented in Table 1.

TABLE 1

| TARGET | TARGET | CONCENTRATION | | MEAN |
|---|---|---|---|---|
| (JMJ) | (KDM) | (M) | READOUT | INHIBITION (%) |
| JMJD1B | KDM3B | 1.0E−05 | Enzymatic activity | 84.87 |
| JMJD1B | KDM3B | 1.0E−06 | Enzymatic activity | 32.63 |
| JMJD1B | KDM3B | 1.0E−07 | Enzymatic activity | 7.088 |
| JMJD1B | KDM3B | 1.0E−08 | Enzymatic activity | 7.050 |
| JARID1B | KDM5B | 1.0E−05 | Enzymatic activity | 57.79 |
| JARID1B | KDM5B | 1.0E−06 | Enzymatic activity | 2.21 |
| JARID1B | KDM5B | 1.0E−07 | Enzymatic activity | 11.75 |
| JARID1B | KDM5B | 1.0E−08 | Enzymatic activity | 6.82 |
| JARID1A | KDM5A | 1.0E−05 | Enzymatic activity | 60.58 |
| JARID1A | KDM5A | 1.0E−06 | Enzymatic activity | 11.63 |
| JARID1A | KDM5A | 1.0E−07 | Enzymatic activity | −0.15 |
| JARID1A | KDM5A | 1.0E−08 | Enzymatic activity | −7.59 |
| JMJD2B | KDM4B | 1.0E−05 | Enzymatic activity | 38.21 |
| JMJD2B | KDM4B | 1.0E−06 | Enzymatic activity | 9.80 |
| JMJD2B | KDM4B | 1.0E−07 | Enzymatic activity | 8.33 |
| JMJD2B | KDM4B | 1.0E−08 | Enzymatic activity | 4.03 |
| JMJD3 | KDM6B | 1.0E−05 | Enzymatic activity | 41.67 |
| JMJD3 | KDM6B | 1.0E−06 | Enzymatic activity | 5.38 |
| JMJD3 | KDM6B | 1.0E−07 | Enzymatic activity | −2.27 |
| JMJD3 | KDM6B | 1.0E−08 | Enzymatic activity | −1.80 |
| PHF8 | KDM7B | 1.0E−05 | Enzymatic activity | 61.29 |
| PHF8 | KDM7B | 1.0E−06 | Enzymatic activity | −15.40 |
| PHF8 | KDM7B | 1.0E−07 | Enzymatic activity | 1.57 |
| PHF8 | KDM7B | 1.0E−08 | Enzymatic activity | −14.20 |
| JMJD1A | KDM3A | 1.0E−05 | Enzymatic activity | 55.09 |
| JMJD1A | KDM3A | 1.0E−06 | Enzymatic activity | 1.35 |
| JMJD1A | KDM3A | 1.0E−07 | Enzymatic activity | 1.90 |
| JMJD1A | KDM3A | 1.0E−08 | Enzymatic activity | −8.56 |
| FBXL10 | KDM2B | 1.0E−05 | Enzymatic activity | 26.26 |
| FBXL10 | KDM2B | 1.0E−06 | Enzymatic activity | −2.61 |
| FBXL10 | KDM2B | 1.0E−07 | Enzymatic activity | −0.81 |
| FBXL10 | KDM2B | 1.0E−08 | Enzymatic activity | −9.52 |
| UTX | KDM6A | 1.0E−05 | Enzymatic activity | 20.45 |
| UTX | KDM6A | 1.0E−06 | Enzymatic activity | −1.25 |
| UTX | KDM6A | 1.0E−07 | Enzymatic activity | 4.06 |
| UTX | KDM6A | 1.0E−08 | Enzymatic activity | −5.08 |

The results at concentrations 1×10⁻⁵ M and 1×10⁻⁶ M are summarized in Table 2.

TABLE 2

| KDM | TARGET | CONC | UNIT | MEASUREMENT | VALUE |
|---|---|---|---|---|---|
| KDM3B | H3K9me1/2(3) | 1.0E−05 | M | Mean % Inhibition | 84.87 |
| KDM5A | H3K4me3 | 1.0E−05 | M | Mean % Inhibition | 60.58 |
| KDM5B | H3K4me3 | 1.0E−05 | M | Mean % Inhibition | 57.79 |
| KDM3A | H3K9me1/2(3) | 1.0E−05 | M | Mean % Inhibition | 55.09 |
| KDM6B | H3K27me3 | 1.0E−05 | M | Mean % Inhibition | 41.67 |
| KDM4B | H3K9me3 | 1.0E−05 | M | Mean % Inhibition | 38.21 |
| KDM | TARGET | CONC | UNIT | MEASUREMENT | VALUE |
| KDM3B | H3K9me1/2(3) | 1.0E−06 | M | Mean % Inhibition | 32.63 |
| KDM5A | H3K4me3 | 1.0E−06 | M | Mean % Inhibition | 11.63 |
| KDM4B | H3K4me3 | 1.0E−06 | M | Mean % Inhibition | 9.80 |
| KDM6B | H3K27me3 | 1.0E−06 | M | Mean % Inhibition | 5.38 |
| KDM5B | H3K4me3 | 1.0E−06 | M | Mean % Inhibition | 2.21 |
| KDM3A | H3K9me3 | 1.0E−06 | M | Mean % Inhibition | 1.35 |

Accordingly, it was observed that PFI-63 inhibited multiple demethylases with the top hit KDM3B (JMJD1B; see Tables 1 and 2) with an IC₅₀ of˜2-3 μM.

### Example 7

This example describes the identification and initial characterization of a second histone demethylase inhibitor, PFI-90.

To search for additional histone demethylase inhibitors, 25 compounds structurally similar were screened in the manner described in Example 2. RH4 ALK-Luc and RH4 CMV-Luc cells in 27 μL medium were seeded into separate white-walled and bottomed 384-well plates. Ten-fold serial dilutions of the 25 test compounds were made using an automated liquid handler, and 3 μL of each dilution was added to each well of plates containing cells. Each test concentration was assayed in quadruplicate. Corresponding DMSO dilutions were used as control. Treated plates were then read following the same procedure used for screening described in Example 1 above. Table 3 presents exemplary data for each tested compound at 10 μM and 1 μM dilutions.

TABLE 3

| | | Avg Absorbance | Avg Absorbance |
|---|---|---|---|
| Compound | Reporter | @ 10 μM | @ 1 μM |
| 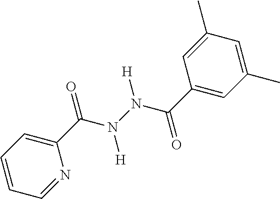 [SMILES: Cc1cc(C)cc(C(=O)NNC(=O)c2ccccn2)c1] PFI-63 | ALK-Luc CMV-Luc | 40.1 74.5 | 91.4 92.3 |
| | ALK-Luc | 89.0 | 90.9 |
| | CMV-Luc | 103.1 | 83.0 |
| 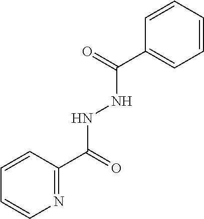 [SMILES: O=C(NNC(=O)c1ccccn1)c1ccccc1] PFI-65 | ALK-Luc CMV-Luc | 109.2 122.1 | 103.5 117.9 |
| 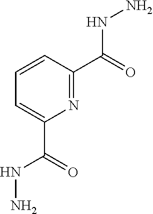 [SMILES: NNC(=O)c1cccc(C(=O)NN)n1] PFI-66 | ALK-Luc CMV-Luc | 91.3 97.0 | 91.6 113.3 |
| 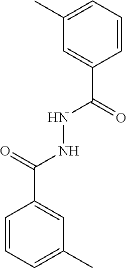 [SMILES: Cc1cccc(C(=O)NNC(=O)c2cccc(C)c2)c1] PFI-67 | ALK-Luc CMV-Luc | 112.8 102.8 | 94.6 107.0 |
| 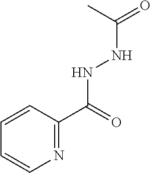 PFI-68 | ALK-Luc CMV-Luc | 98.0 105.4 | 95.0 107.2 |
| 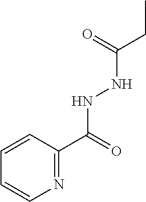 PFI-69 | ALK-Luc CMV-Luc | 94.4 98.5 | 104.3 100.3 |
| 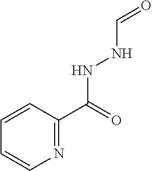 [SMILES: O=CNNC(=O)c1ccccn1] PFI-70 | ALK-Luc CMV-Luc | 93.3 103.6 | 99.7 108.0 |
| 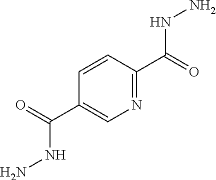 [SMILES: NNC(=O)c1ccc(C(=O)NN)nc1] PFI-71 | ALK-Luc CMV-Luc | 108.6 101.4 | 100.1 100.9 |
| 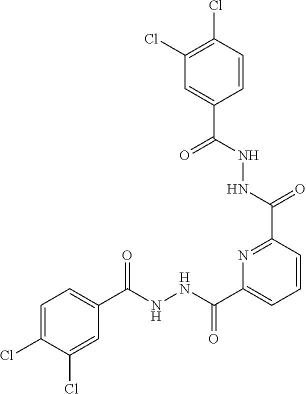 [SMILES: O=C(NNC(=O)c1cccc(C(=O)NNC(=O)c2ccc(Cl)c(Cl)c2)n1)c1ccc(Cl)c(Cl)c1] PFI-72 | ALK-Luc CMV-Luc | 91.1 87.7 | 97.2 97.2 |
| 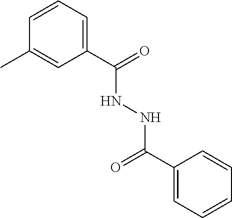 [SMILES: Cc1cccc(C(=O)NNC(=O)c2ccccc2)c1] PFI-73 | ALK-Luc CMV-Luc | 122.8 119.4 | 98.3 113.2 |
| 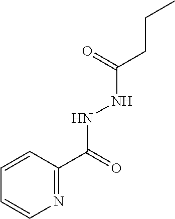 PFI-74 | ALK-Luc CMV-Luc | 94.7 103.9 | 102.4 101.0 |
| 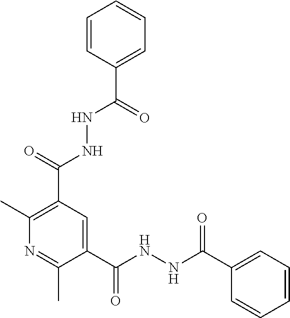 [SMILES: Cc1nc(C)c(C(=O)NNC(=O)c2ccccc2)cc1C(=O)NNC(=O)c1ccccc1] PFI-75 | ALK-Luc CMV-Luc | 110.5 104.9 | 100.2 96.5 |
| 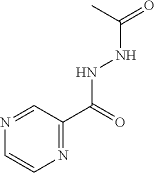 PFI-76 | ALK-Luc CMV-Luc | 100.2 102.5 | 105.6 97.9 |
| 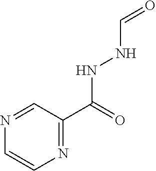 [SMILES: O=CNNC(=O)c1cnccn1] PFI-77 | ALK-Luc CMV-Luc | 96.5 106.1 | 95.8 99.0 |
| 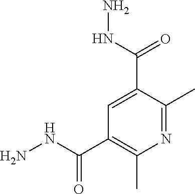 [SMILES: Cc1nc(C)c(C(=O)NN)cc1C(=O)NN] PFI-78 | ALK-Luc CMV-Luc | 95.3 104.3 | 100.3 95.6 |
| 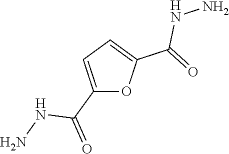 [SMILES: NNC(=O)c1ccc(C(=O)NN)o1] PFI-79 | ALK-Luc CMV-Luc | 97.6 101.5 | 97.0 95.0 |
| 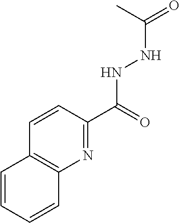 [SMILES: CC(=O)NNC(=O)c1ccc2ccccc2n1] PFI-80 | ALK-Luc CMV-Luc | 96.6 90.7 | 90.1 105.4 |
| 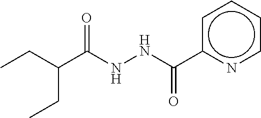 PFI-81 | ALK-Luc CMV-Luc | 89.1 86.1 | 97.7 105.1 |
| 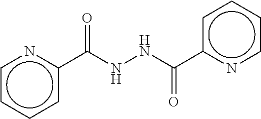 PFI-82 | ALK-Luc CMV-Luc | 107.1 136.3 | 112.4 103.8 |
| 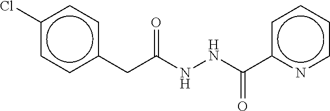 PFI-83 | ALK-Luc CMV-Luc | 32.2 90.6 | 97.8 107.2 |
| 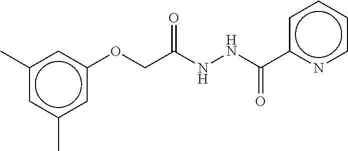 PFI-84 | ALK-Luc CMV-Luc | 56.5 74.8 | 99.4 100.8 |
| 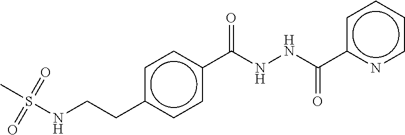 PFI-85 | ALK-Luc CMV-Luc | 100.2 108.9 | 99.7 101.7 |
| 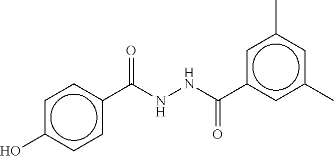 PFI-86 | ALK-Luc CMV-Luc | 84.1 104.7 | 97.7 92.6 |
| 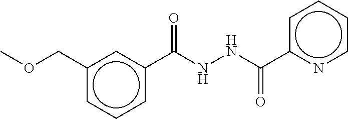 PFI-87 | ALK-Luc CMV-Luc | 120.4 151.1 | 115.5 123.7 |
| 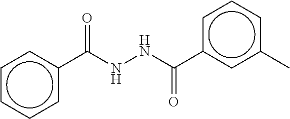 PFI-88 | ALK-Luc CMV-Luc | 121.4 118.7 | 91.6 102.8 |
| 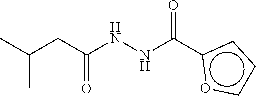 PFI-89 | ALK-Luc CMV-Luc | 92.5 98.3 | 95.1 97.7 |
| 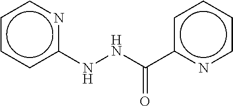 PFI-90 | ALK-Luc CMV-Luc | 34.0 160.9 | 55.9 78.0 |

The screen identified a second potential histone demethylase inhibitor, designated PFI-90.

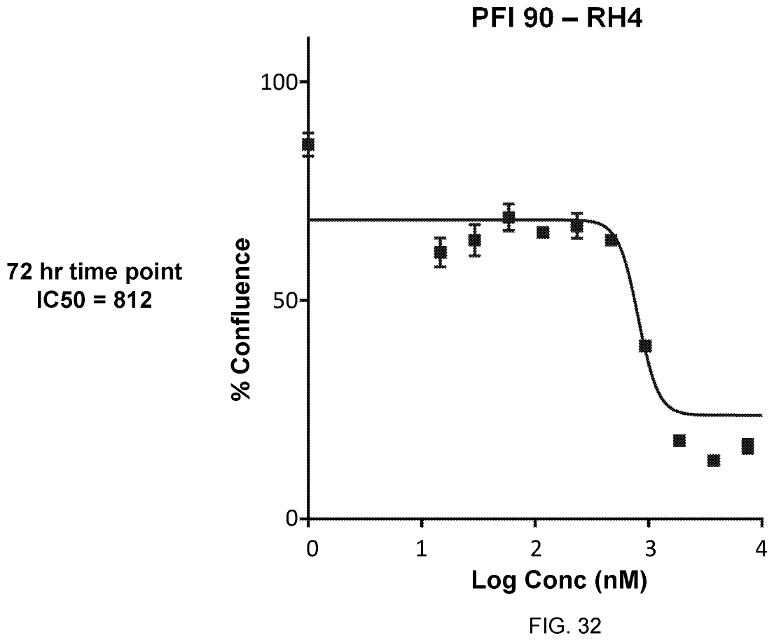

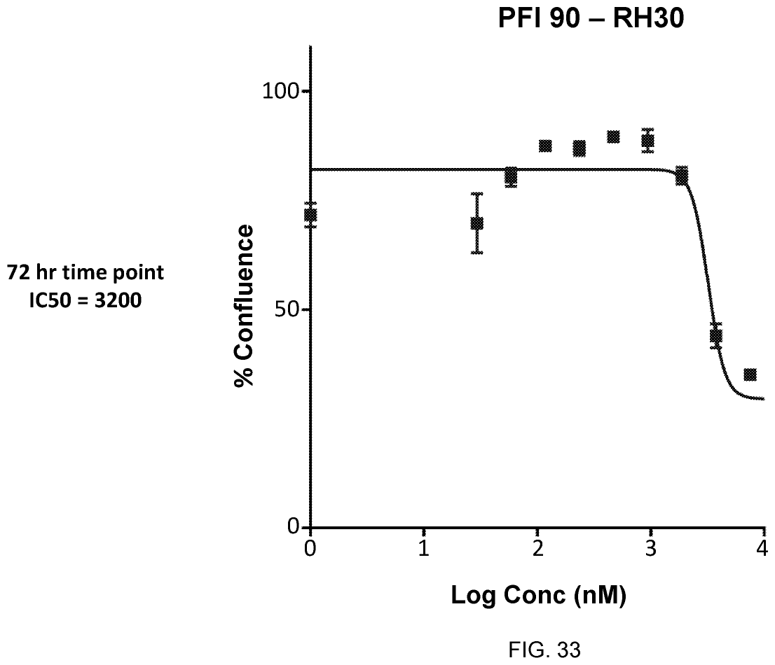

A cell confluence assay was performed to assess the cytotoxicity of PFI-90 in RH4 and RH30 rhabdomyosarcoma cells. Confluence data was collected via photographic images of cells treated with PFI-90 at the following time points: 24 hours, 48 hours, 72 hours, 96 hours and 120 hours. Exemplary dose response curves at 72 hours are presented as FIGS. 32 and 33 in RH4 and RH30 cells, respectively. The results indicate that PFI-90 is more cytotoxic to rhabdomyosarcoma cells than PFI-63.

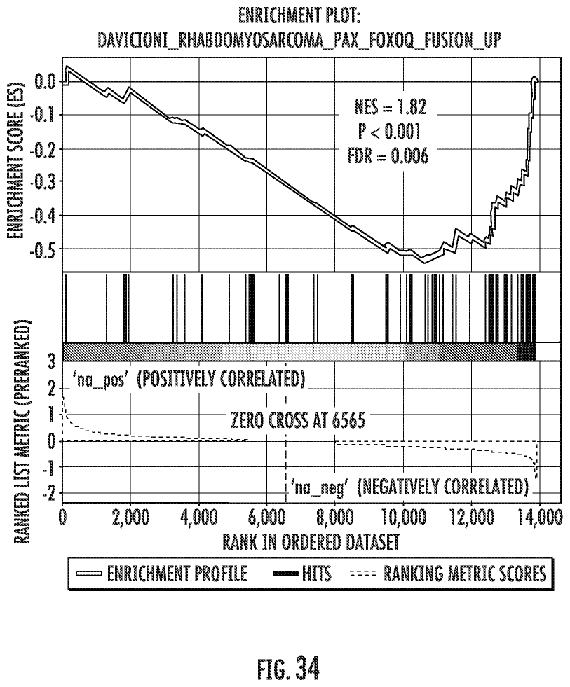

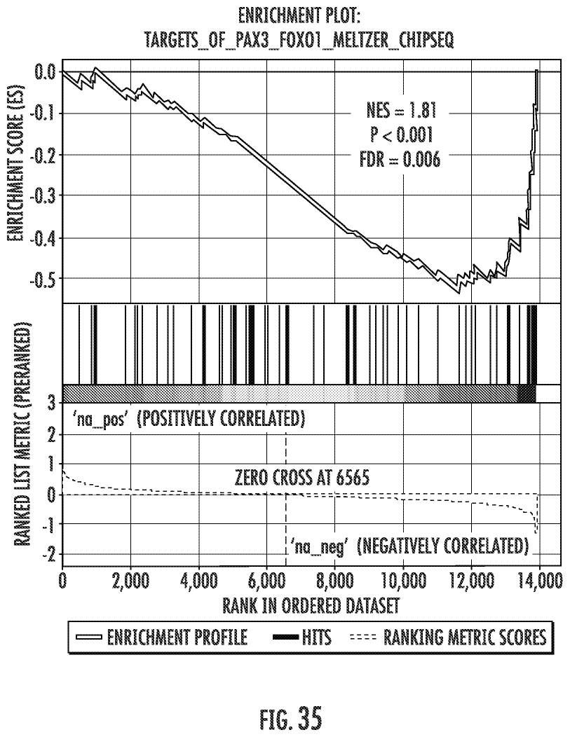

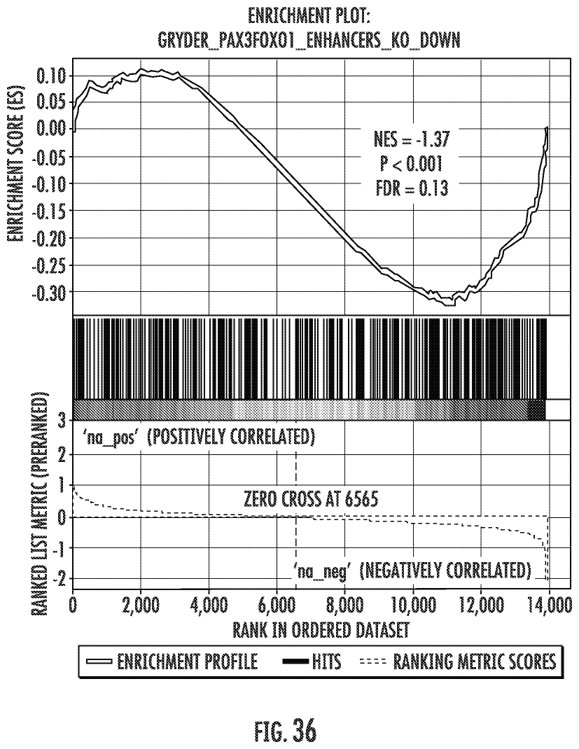

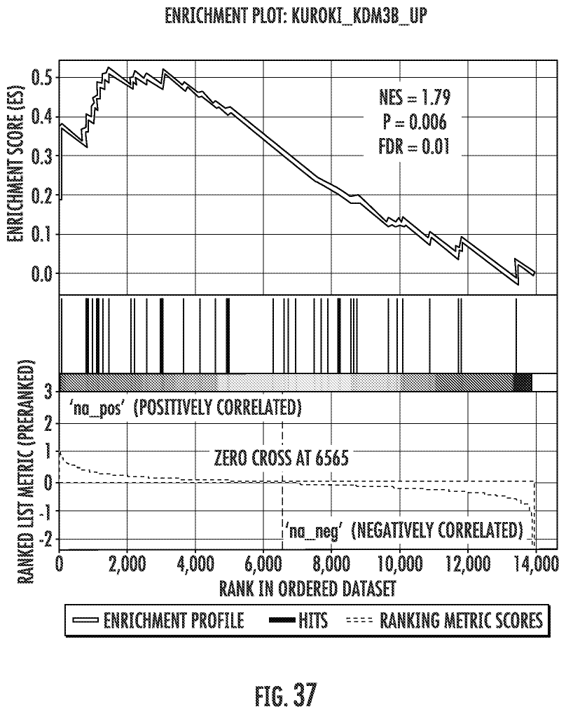

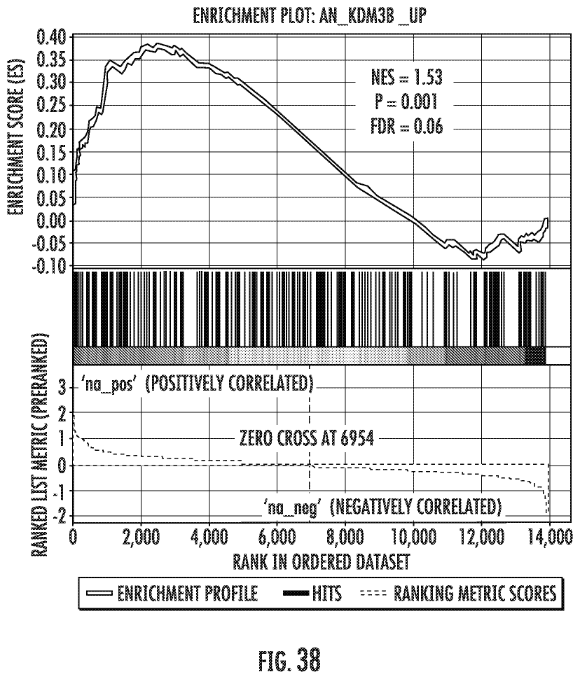

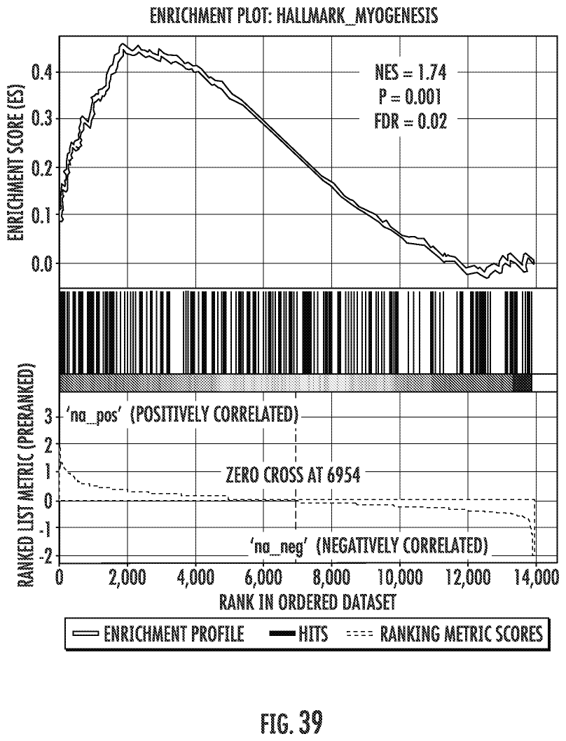

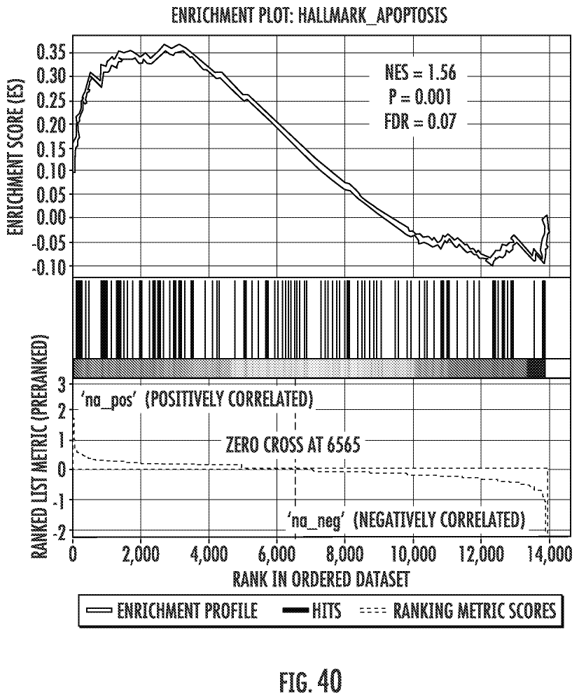

GSEA assays like those performed in Example 3 above, were also performed to assess PFI-90. The results for PFI-90 were similar to those obtained for PFI-63. Targets of the PAX3-FOXO1 fusion transcription factor are downregulated in RH4 cells in the presence of PFI-90 (see FIGS. 34-36). The results also indicate that genes that are induced upon KDM3B suppression are upregulated in RH4 cells treated with PFI-90 (see FIGS. 37-38). The results also indicate that cells treated with PFI-90 experience upregulation of targets associated with myogenic differentiation and apoptosis (see FIGS. 39-40).

PFI-90 also has improved solubility relative to PFI-63, with a Log P value of 0.82 compared with a Log P value of 2.36 for PFI-63.

### Example 8

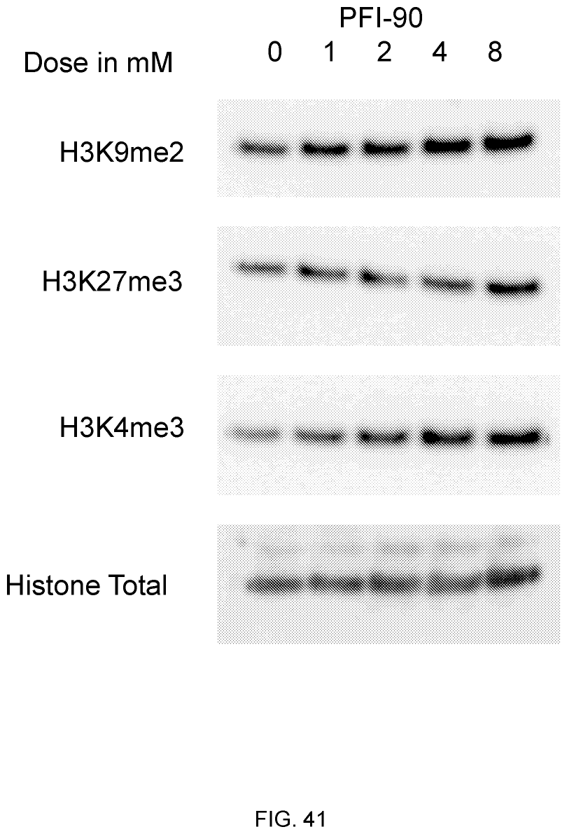

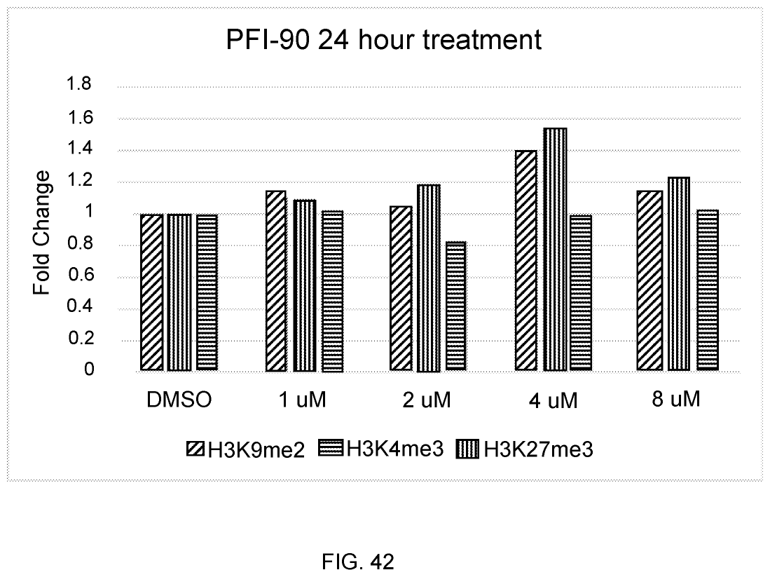

Analysis via Western blot confirms that PFI-90 inhibits lysine demethylase activity. FIG. 41 shows that as the concentration of PFI-90 increases, the methylation of histone H3 also increases. These results are summarized in FIG. 42.

As described above, PFI-90 inhibitory activity was determined by Eurofins using enzymatic inhibition activity analysis of a panel of histone demethylases. The results are presented in Table 4.

TABLE 4

| TARGET | TARGET | CONCENTRATION | | MEAN |
|---|---|---|---|---|
| (JMJ) | (KDM) | (M) | READOUT | INHIBITION (%) |
| JMJD1B | KDM3B | 1.0E−05 | Enzymatic activity | 87.14 |
| JMJD1B | KDM3B | 5.0E−06 | Enzymatic activity | 81.56 |
| JMJD1B | KDM3B | 2.5E−06 | Enzymatic activity | 70.58 |
| JMJD1B | KDM3B | 1.3E−06 | Enzymatic activity | 55.46 |
| JMJD1B | KDM3B | 6.3E−07 | Enzymatic activity | 36.89 |
| JMJD1B | KDM3B | 3.1E−07 | Enzymatic activity | 30.81 |
| JMJD1B | KDM3B | 1.6E−07 | Enzymatic activity | 24.47 |
| JMJD1B | KDM3B | 7.8E−08 | Enzymatic activity | 14.32 |
| JARID1A | KDM5A | 1.0E−05 | Enzymatic activity | 83.32 |
| JARID1A | KDM5A | 5.0E−06 | Enzymatic activity | 64.60 |
| JARID1A | KDM5A | 2.5E−06 | Enzymatic activity | 51.29 |
| JARID1A | KDM5A | 1.3E−06 | Enzymatic activity | 20.23 |
| JARID1A | KDM5A | 6.3E−07 | Enzymatic activity | 15.42 |
| JARID1A | KDM5A | 3.1E−07 | Enzymatic activity | 7.18 |
| JARID1A | KDM5A | 1.6E−07 | Enzymatic activity | 2.33 |
| JARID1A | KDM5A | 7.8E−08 | Enzymatic activity | −10.90 |
| JMJD2B | KDM4B | 1.0E−05 | Enzymatic activity | 82.94 |
| JMJD2B | KDM4B | 5.0E−06 | Enzymatic activity | 71.59 |
| JMJD2B | KDM4B | 2.5E−06 | Enzymatic activity | 52.51 |
| JMJD2B | KDM4B | 1.3E−06 | Enzymatic activity | 38.48 |
| JMJD2B | KDM4B | 6.3E−07 | Enzymatic activity | 27.94 |
| JMJD2B | KDM4B | 3.1E−07 | Enzymatic activity | 11.55 |
| JMJD2B | KDM4B | 1.6E−07 | Enzymatic activity | 16.27 |
| JMJD2B | KDM4B | 7.8E−08 | Enzymatic activity | 41.56 |
| JMJD3 | KDM6B | 1.0E−05 | Enzymatic activity | 46.80 |
| JMJD3 | KDM6B | 5.0E−06 | Enzymatic activity | 17.41 |
| JMJD3 | KDM6B | 2.5E−06 | Enzymatic activity | 11.55 |
| JMJD3 | KDM6B | 1.3E−06 | Enzymatic activity | 8.42 |
| JMJD3 | KDM6B | 6.3E−07 | Enzymatic activity | 6.81 |
| JMJD3 | KDM6B | 3.1E−07 | Enzymatic activity | −0.60 |
| JMJD3 | KDM6B | 1.6E−07 | Enzymatic activity | 25.15 |
| JMJD3 | KDM6B | 7.8E−08 | Enzymatic activity | 8.60 |

The results at concentrations 1×10⁻⁵ M and 1×10⁻⁶ M are summarized in Table 5.

TABLE 5

| KDM | TARGET | CONC | UNIT | MEASUREMENT | VALUE |
|---|---|---|---|---|---|
| KDM3B | H3K9me1/2(3) | 1.0E−05 | M | Mean % Inhibition | 87.1 |
| KDM5A | H3K4me3 | 1.0E−05 | M | Mean % Inhibition | 83.3 |
| KDM4B | H3K9me3 | 1.0E−05 | M | Mean % Inhibition | 82.9 |
| KDM6B | H3K27me3 | 1.0E−05 | M | Mean % Inhibition | 46.8 |
| KDM | TARGET | CONC | UNIT | MEASUREMENT | VALUE |
| KDM3B | H3K9me1/2(3) | 1.0E−06 | M | Mean % Inhibition | 55.5 |
| KDM4B | H3K4me3 | 1.0E−06 | M | Mean % Inhibition | 38.5 |
| KDM5A | H3K4me3 | 1.0E−06 | M | Mean % Inhibition | 20.2 |
| KDM6B | H3K27me3 | 1.0E−06 | M | Mean % Inhibition | 8.4 |

Accordingly, it was observed that PFI-90 inhibited multiple demethylases with the top hit KDM3B (JMJD1B; see Table 5).

### Example 9

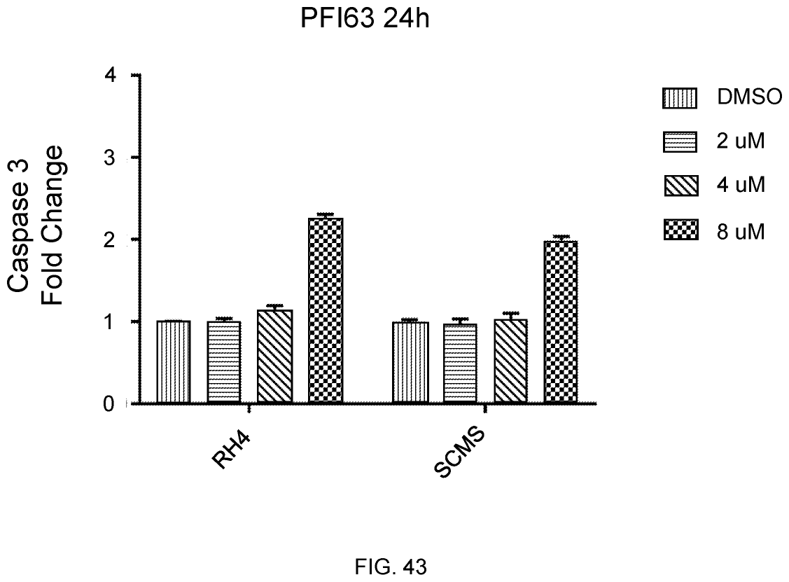

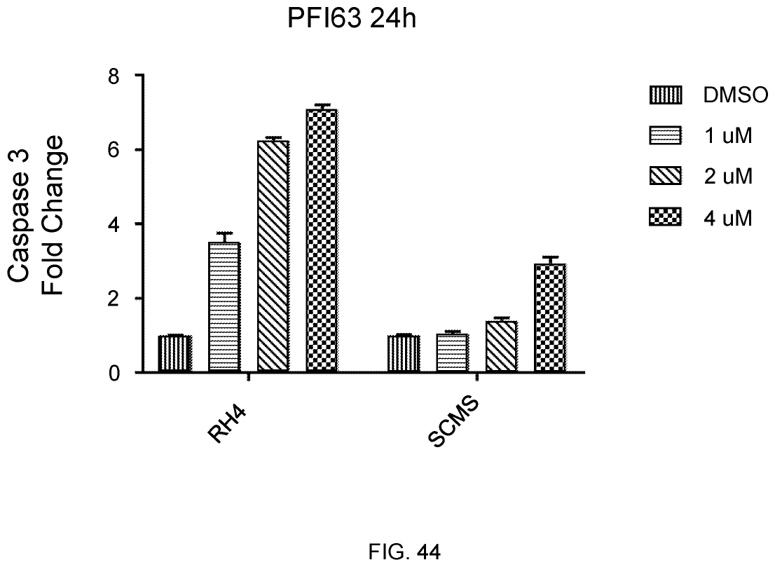

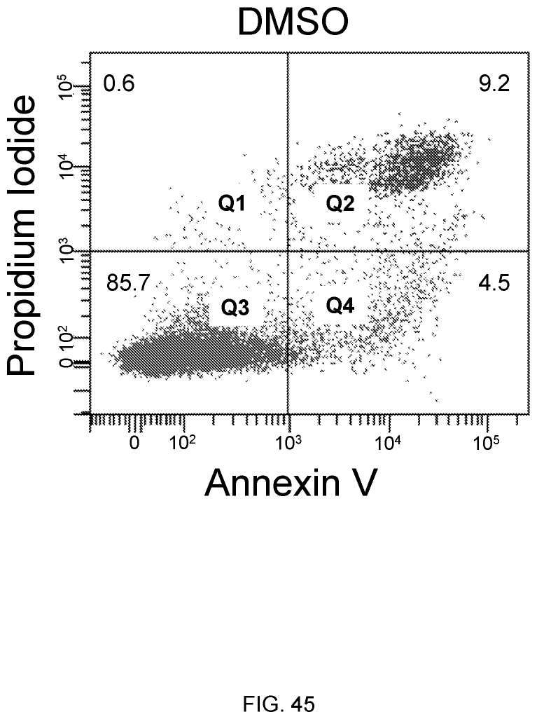

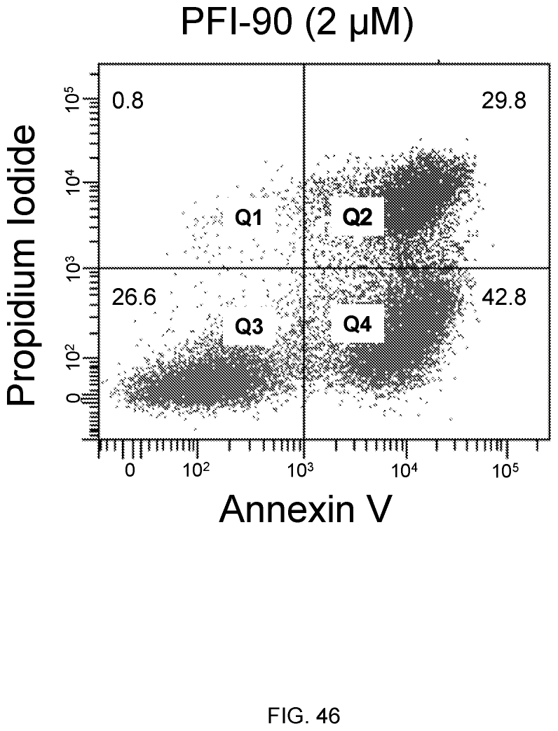

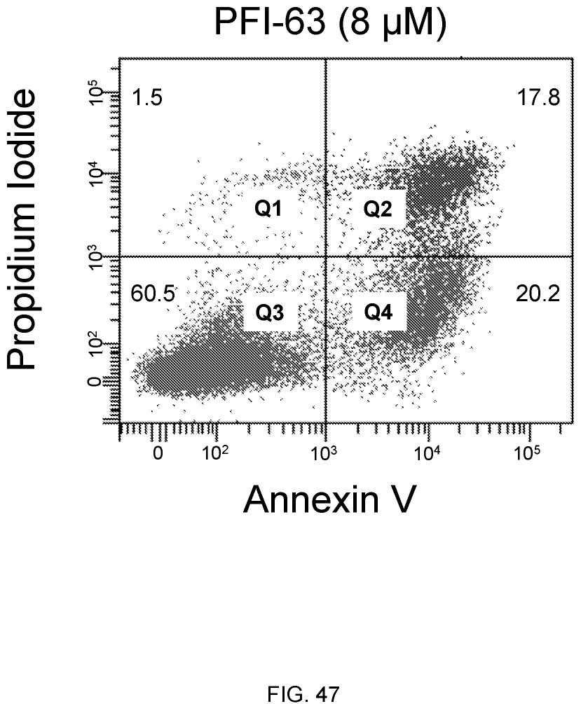

The impact of PFI-63 and PFI-90 on apoptosis in rhabdomyosarcoma cells was investigated by assaying caspase 3 levels in the cells, and by annexin V staining. In both RH4 and SCMC human rhabdomyosarcoma cells, PFI-63 and PFI-90 lead to increased caspase 3 (FIGS. 43 and 44). Annexin staining indicates an increase in cell death relative to DMSO control (FIG. 45) for RH4 cells treated with PFI-90 (FIG. 46) and PFI-63 (FIG. 47).

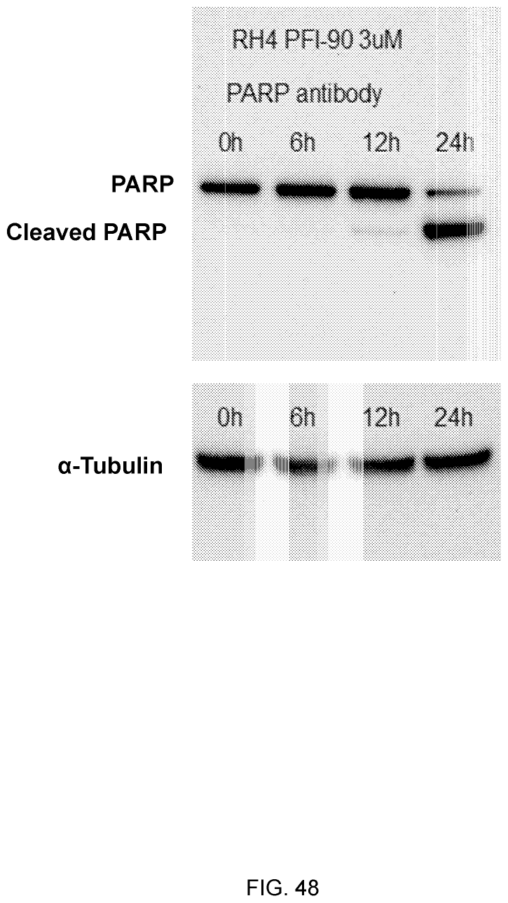

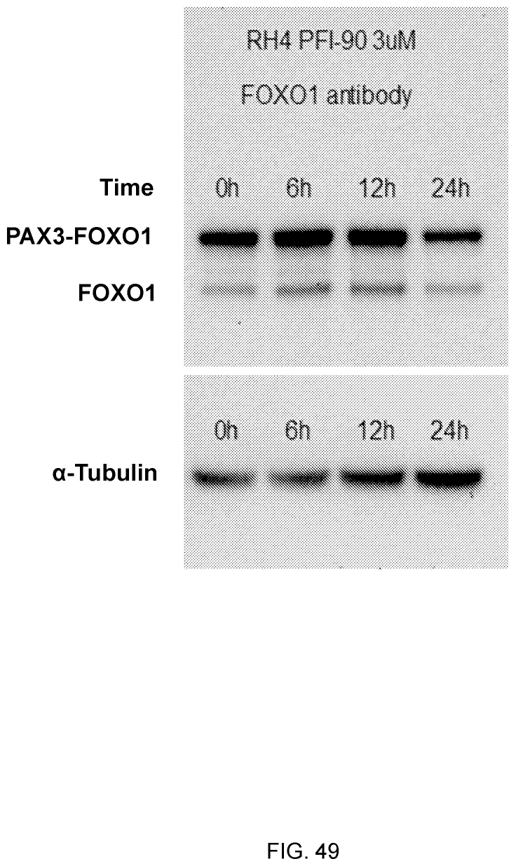

Analysis via Western Blot also shows an increase in apoptosis. In RH4 cells treated with PFI-90 at 3 μM, an increase in cleaved PARP (a marker for apoptosis) is observed at 24 hours (FIG. 48). This coincides with a decrease in PAX3-FOXO1 which is also observed at 24 hours (FIG. 49).

This example further demonstrates that PFI-63 and PFI-90 increase apoptosis in RH4 and SCMC cells.

### Example 10

This example describes the testing of PFI-90 against the NCI-60 panel. The NCI-60 cancer cell line panel is a collection of 60 human cancer cell lines maintained by the National Cancer Institute (see Shoemaker et al., Nat. Rev. Cancer, 6: 813-823 (2006)). Briefly, the percentage growth of the cancer cells lines was measured in the presence of PFI-90 at various concentrations. Results indicate that PFI-90 has the potential to treat a range of cancers.

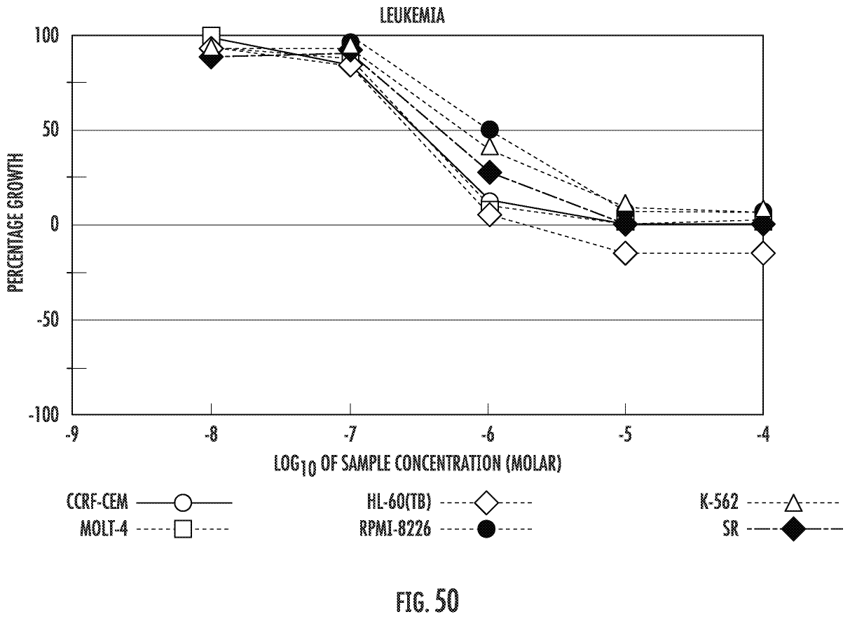

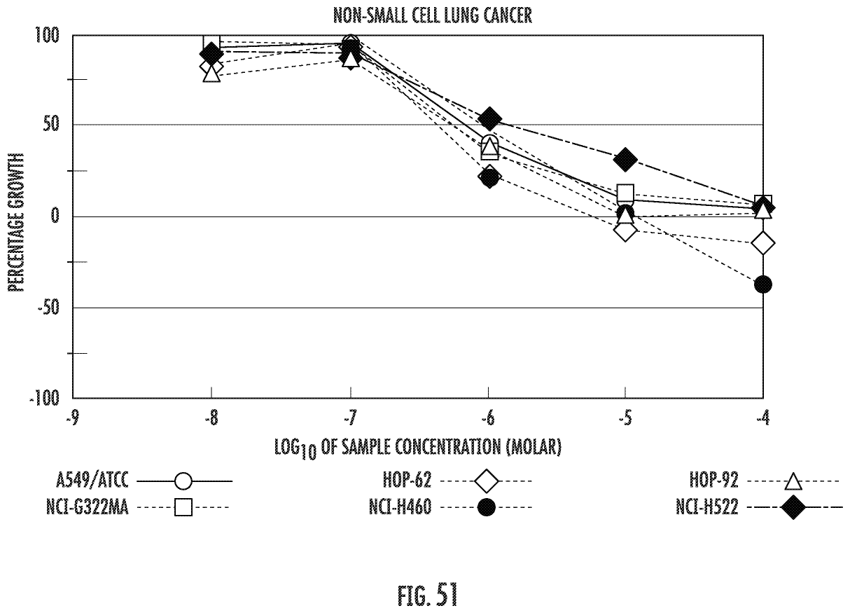

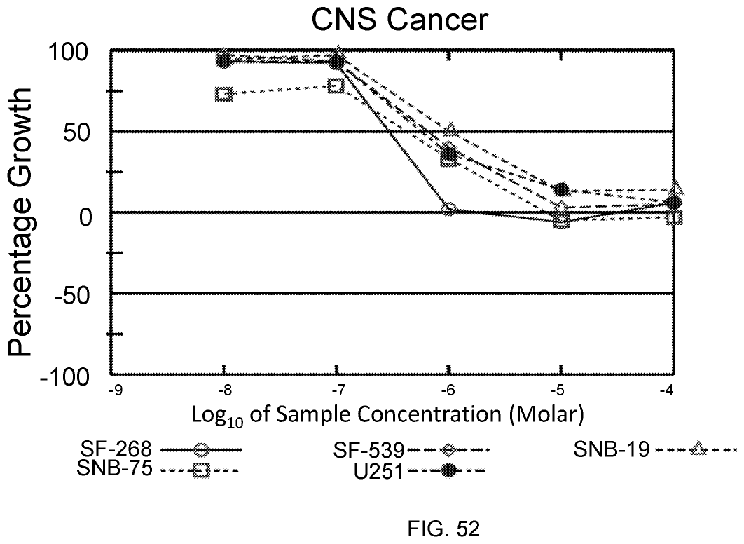

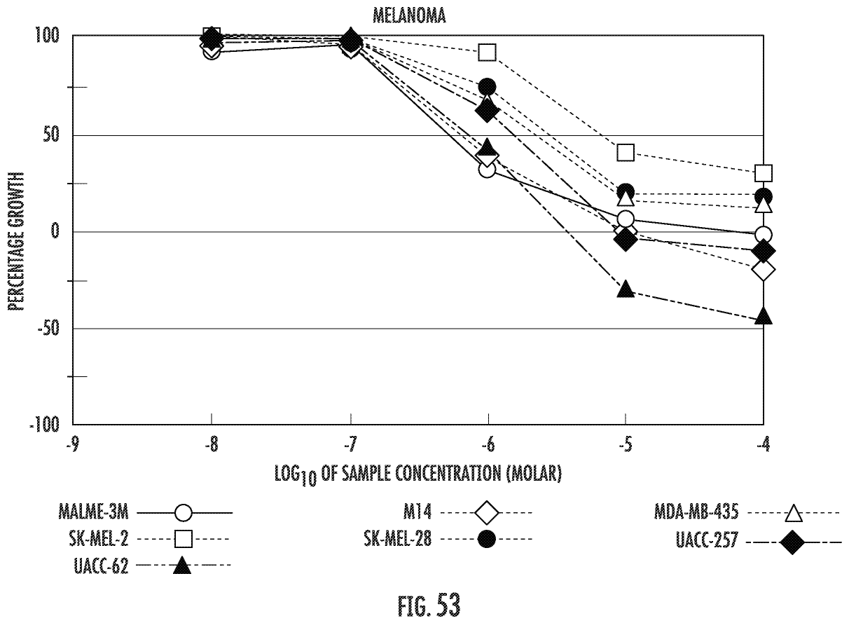

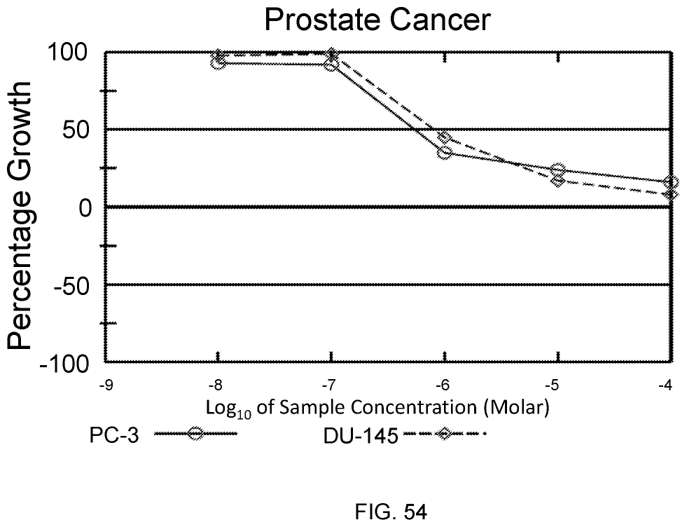

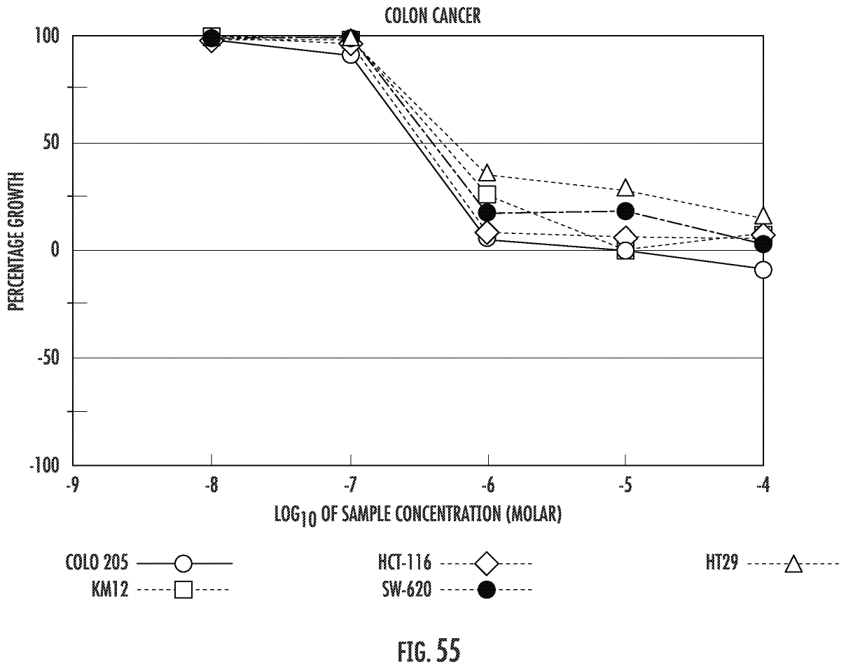

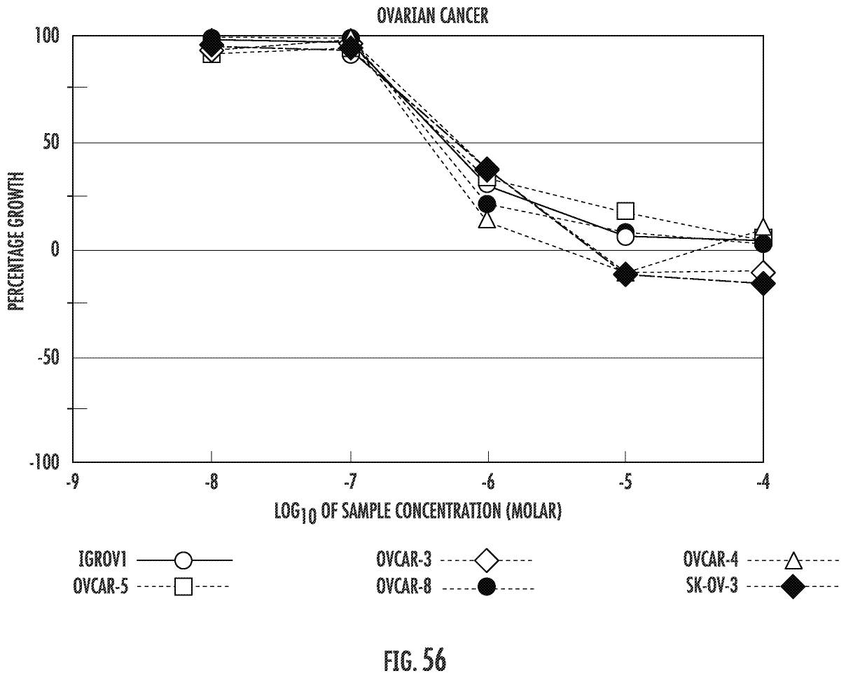

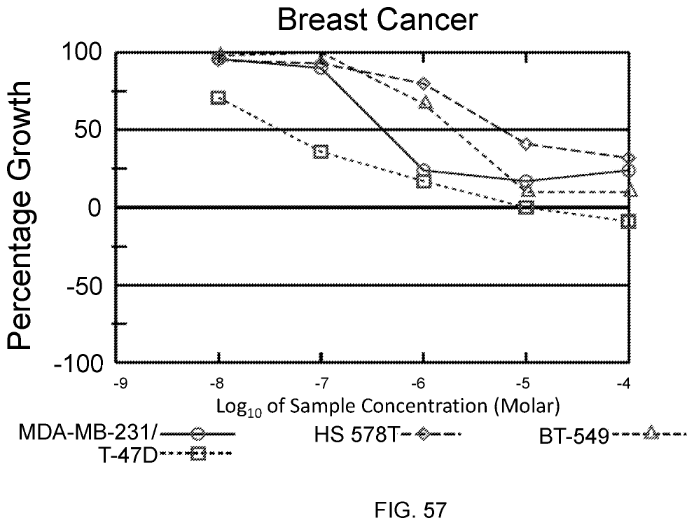

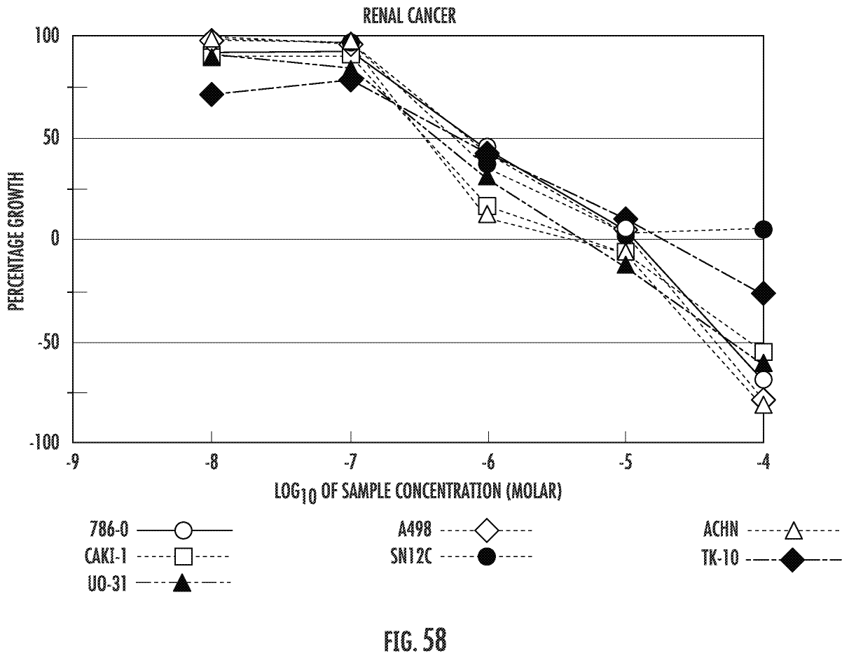

FIG. 50 shows PFI-90's inhibition of various leukemia cell lines. At higher concentrations PFI-90 slows and/or stops the growth of the cells. Similar results were obtained for non-small cell lung cancers (FIG. 51), CNS cancers (FIG. 52), melanoma (FIG. 53), prostate cancer (FIG. 54), colon cancer (FIG. 55), ovarian cancer (FIG. 56) and breast cancers (FIG. 57). In renal cancer cell lines, PFI-90 inhibits growth and leads to cell death (FIG. 58).

### Example 11

This example describes testing of PFI-63 and PFI-90 in Ewing's sarcoma and osteosarcoma cells.

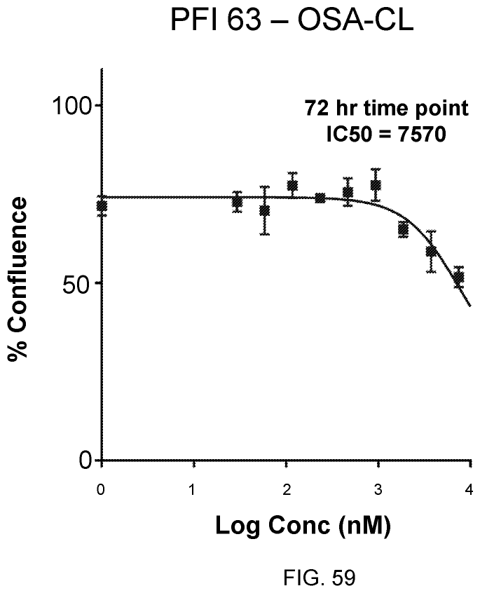

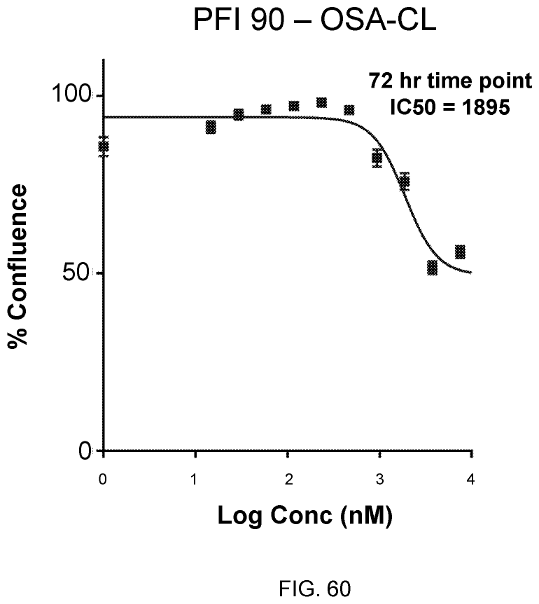

A cell confluence assay was performed to further assess the cytotoxicity of PFI-63 in in OSA-CL osteosarcoma cells. Confluence data was collected via photographic images of cells treated with PFI-63 at the following time points: 24 hours, 48 hours, 72 hours, 96 hours and 120 hours. Exemplary dose response curves at 72 hours are presented as FIGS. 59 and 60 for PFI-63 and PFI-90 respectively. The results indicate that PFI-63 is toxic to osteosarcoma cells with an IC₅₀ of approximately 7.5 μM. The results indicate that PFI-90 is toxic to osteosarcoma cells with an IC₅₀ of approximately 2 μM.

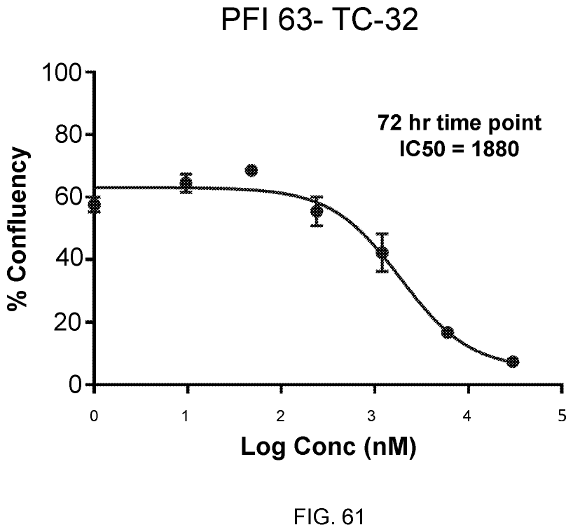

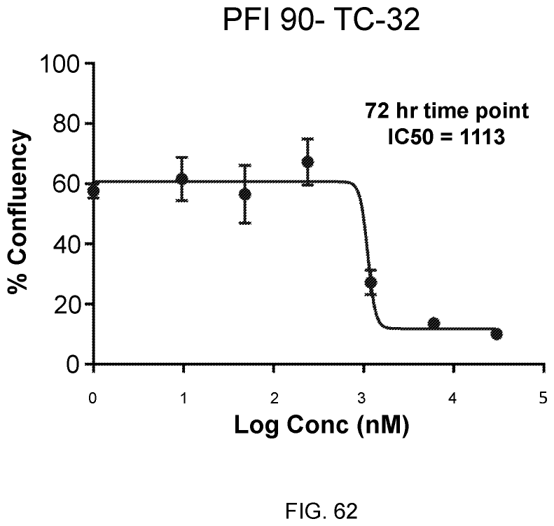

A similar assay was performed to assess the cytotoxicity of PFI-63 and PFI-90 in TC-32 Ewing's sarcoma cells. Exemplary dose response curves at 72 hours are presented as FIGS. 61 and 62 for PFI-63 and PFI-90 respectively. The results indicate that PFI-63 is toxic to Ewing's sarcoma cells with an IC₅₀ of approximately 2 μM. The results indicate that PFI-90 is toxic to Ewing's sarcoma cells with an IC₅₀ of approximately 1 μM.

This example indicates that PFI-90 and PFI-63 are cytotoxic to Ewing's sarcoma and osteosarcoma cells.

### Example 12

Nuclear magnetic resonance (NMR) binding assays confirmed specific binding of PFI-90 to the catalytic domain of KDM3B. A WaterLOGSY assay and a Carr-Purcell Meiboom-Gill (CPMG) based assay were used to investigate protein-ligand binding affinity between PFI-90 and a truncated KDM3B catalytic domain.

WaterLOGSY (Water-Ligand Observed via Gradient Spectroscopy) is a Proton NMR experiment used in drug-discovery to validate the presence of a reversible binding interaction of mild to high affinity from a small molecule to a label-free target (e.g., a protein). In traditional proton NMR experiments, a series of peaks are generated that represent the structure of a solitary small molecule, whereas the WaterLOGSY experiment examines the behavior of a small molecule's peaks in the presence of a potential target. This is achieved by observing differences in interaction between a small molecule interacting with water co-occupying a binding cleft on a target as compared to water dispersed freely in solution. The experiment then depicts the ratio of bound to unbound small molecule by phasing proton NMR spectra on the X-axis in opposing directions depending on the magnitude of that ratio (i.e., increasing ratios above 1 will phase positively, decreasing ratios below 1 will phase negatively).

CPMG (Carr-Purcell-Meiboom-Gill) Relaxation Editing Proton NMR is another drug-discovery NMR experiment that may be used in conjunction with WaterLOGSY to orthogonally validate the presence of a reversible binding interaction of mild to high affinity from a small molecule to a label-free target. In contrast to WaterLOGSY, all peaks are phased above the X-axis. Instead, this experiment filters out the proportion of peak height that is contributed from the quantity of small molecule bound to a target of high molecular weight (e.g., a protein). Once the attenuation of peak height is equivalent to a 20% reduction, the compound is said to bind, with an increasing ratio of bound to unbound small molecules manifesting in increasing signal attenuation.

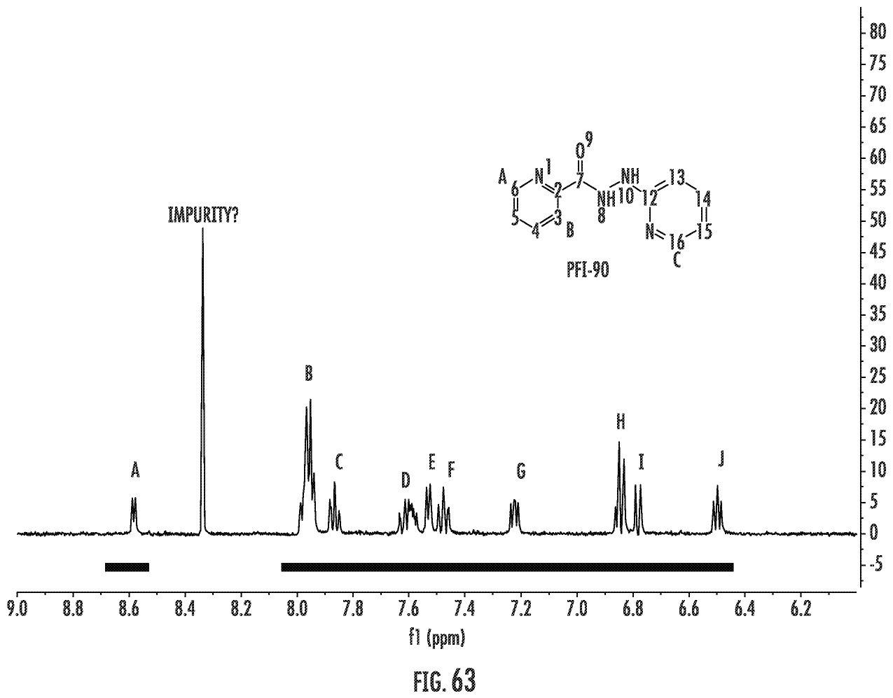

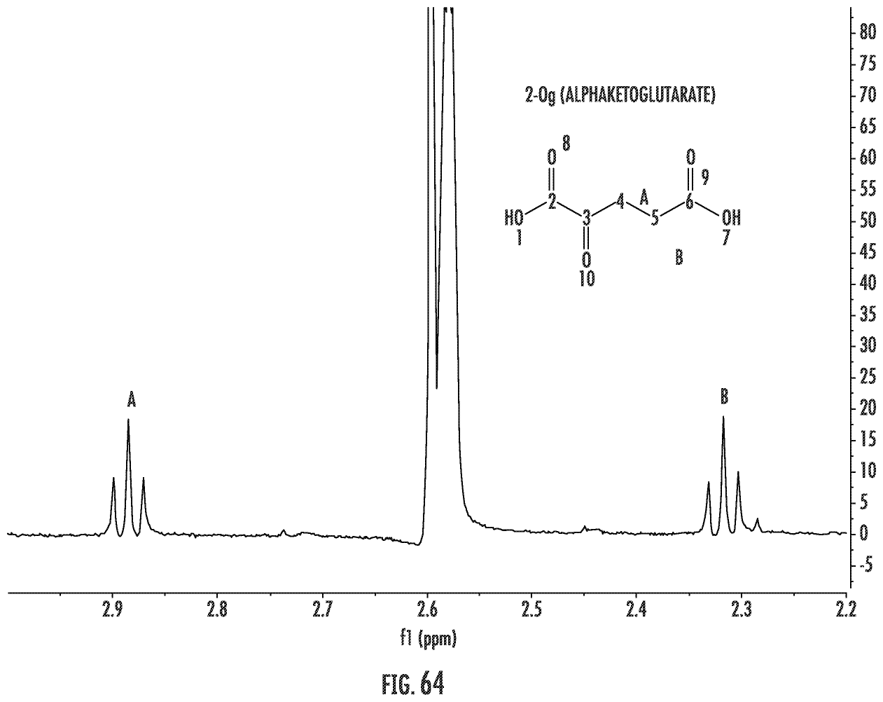

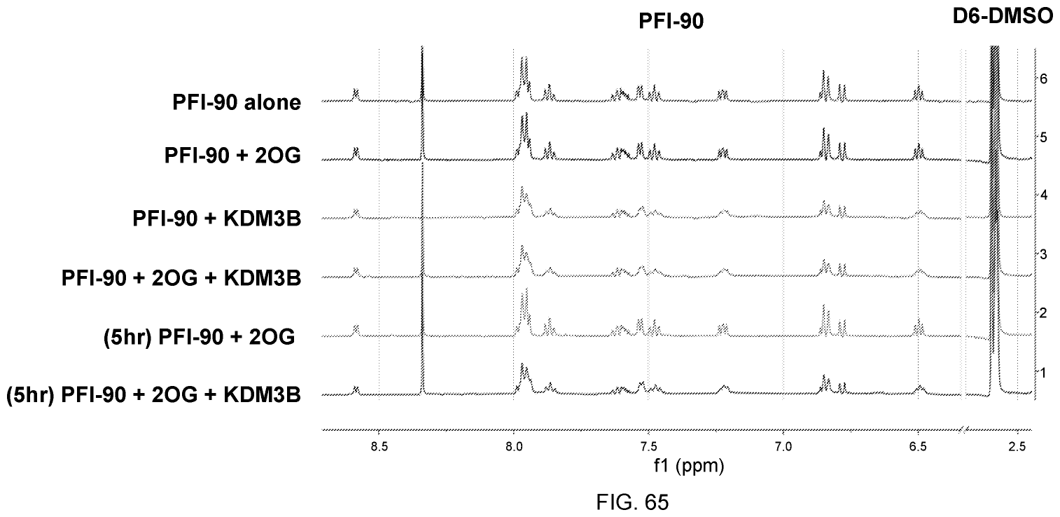

For reference, FIG. 63 depicts NMR peaks for PFI-90. While the peaks are complex, all relevant peaks are clearly resolved. FIG. 64 depicts NMR peaks obtained for uketoglutarate (also called 2-oxoglutaric acid or “2-OG”), a substrate of histone demethylases. 2-OG peaks are easily resolved. FIG. 65 depicts results from a 1D 1H analysis of PFI-90. Chemical shift perturbation upon addition of the truncated KDM3B catalytic domain are not influences by 2-OG presence.

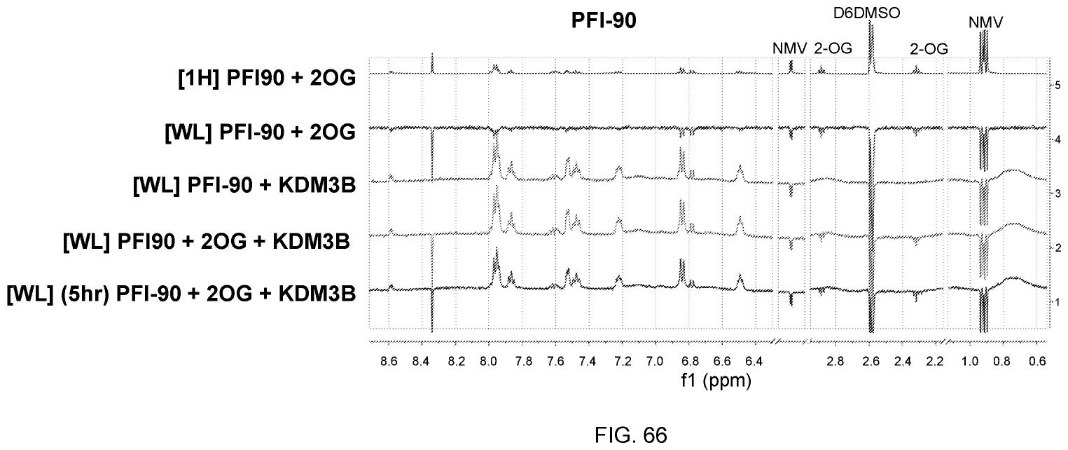

In the WaterLOGSY experiment, PFI-90 was found to bind at 300 μM in the presence and in the absence of 2-OG. 2-OG did not bind at 100 μM (see FIG. 66).

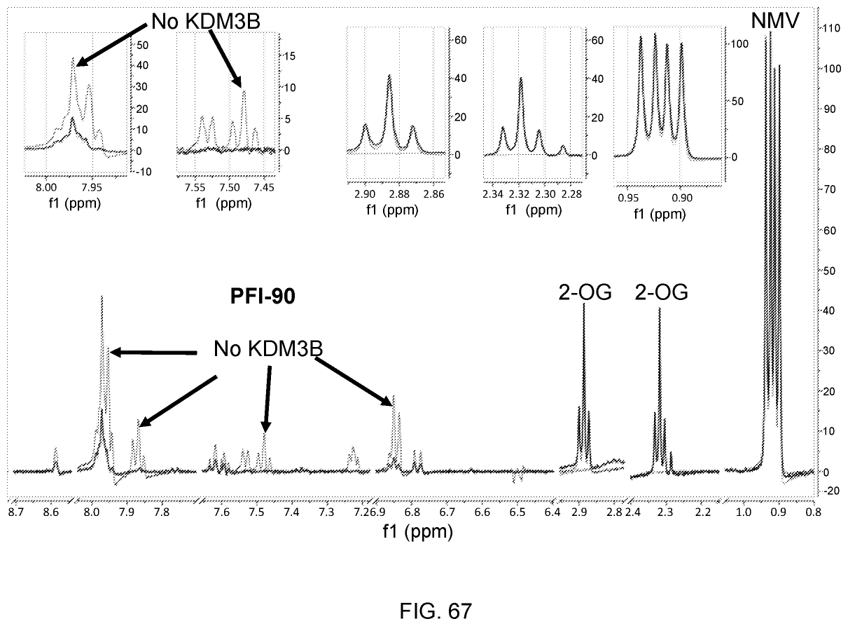

In the CPMG experiment, strong binding of PFI-90 was observed in the presence and absence of 2-OG. 2-OG did not bind at 100 μM. Complete/near complete height attenuation is observed in 5 out of 9 PFI-90 peaks, 70% attenuation is observed in 1 of 9 peaks and 50% attenuation is observed in 3 of 9 peaks (see FIG. 67).

These NMR assays established that PFI-90 binds to the truncated KDM3B catalytic domain.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A pharmaceutical composition comprising (a) one or more compounds selected from the group consisting of 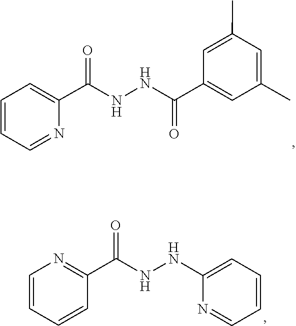 [SMILES: Cc1cc(C)cc(C(=O)NNC(=O)c2ccccn2)c1 ; O=C(NNc1ccccn1)c1ccccn1]
and pharmaceutically acceptable salts thereof, and (b) and a pharmaceutically acceptable carrier.

2.-8. (canceled)

9. A method of treating cancer in a mammal, comprising administering an effective amount of one or more compounds selected from the group consisting of: 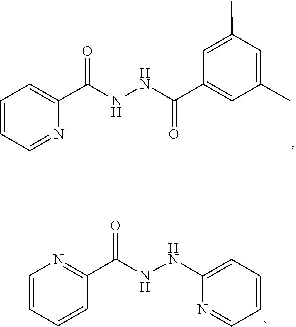 [SMILES: Cc1cc(C)cc(C(=O)NNC(=O)c2ccccn2)c1 ; O=C(NNc1ccccn1)c1ccccn1]
and pharmaceutically acceptable salts thereof, to the mammal.

10. The method of claim 9, wherein the compound is:

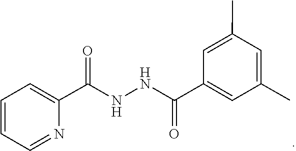 [SMILES: Cc1cc(C)cc(C(=O)NNC(=O)c2ccccn2)c1]

11. The method of claim 9, wherein the compound is:

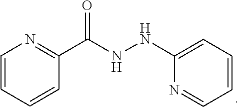 [SMILES: O=C(NNc1ccccn1)c1ccccn1]

12. The method of claim 9, wherein the cancer is a transcription driven cancer.

13. The method of claim 9, wherein the cancer is selected from the group consisting of rhabdomyosarcoma, Ewing's sarcoma, colorectal cancer, ovarian cancer, prostate cancer, CNS cancer, melanoma, breast cancer, leukemia, non-small cell lung cancer, renal cancer, and osteosarcoma.

14. The method of claim 9, wherein the cancer is Ewing's sarcoma.

15. The method of claim 9, wherein the cancer is rhabdomyosarcoma.

16. The method of claim 15, wherein the cancer is alveolar rhabdomyosarcoma.

17. The method of claim 15, wherein the cancer is selected from the group consisting of PAX3-FOXO1 fusion transcription factor positive rhabdomyosarcoma and PAX7-FOXO1 fusion transcription factor positive rhabdomyosarcoma.

18. The method of claim 9, wherein a histone demethylase is inhibited by the compound in the mammal.

19. The method of claim 18, wherein the histone demethylase is a lysine-specific histone demethylase.

20. The method of claim 19, wherein the lysine specific histone demethylase is selected from the group consisting of KDM3A, KDM3B, KDM5A, KDM5B, KDM4B, and KDM6B.

21. The method of claim 20, wherein the histone demethylase is KDM3B.

22. The method of claim 17, wherein the cancer is PAX3-FOXO1 fusion transcription factor positive rhabdomyosarcoma, and wherein the function of the PAX3-FOXO1 fusion transcription factor is disrupted by the compound in the mammal.

23. The method of claim 17, wherein the cancer is PAX7-FOXO1 fusion transcription factor positive rhabdomyosarcoma, and wherein the function of the PAX7-FOXO1 fusion transcription factor is disrupted by the compound in the mammal.

24. A method for inhibiting a histone demethylase in cells comprising contacting the cells with one or more compounds selected from the group consisting of: 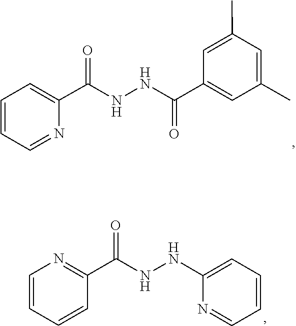 [SMILES: Cc1cc(C)cc(C(=O)NNC(=O)c2ccccn2)c1 ; O=C(NNc1ccccn1)c1ccccn1]
and pharmaceutically acceptable salts thereof.

25.-39. (canceled)
